(19) United States
(12) Patent Application Publication
(10) Pub. No.: US 2014/0155768 A1
(43) Pub. Date: Jun. 5, 2014

(54) Method and Systems for Delivering and Deploying a Sensory Implant In Situ

(71) Applicant: Vectorious Medical Technologies Ltd., Tel-Aviv (IL)

(72) Inventors: Eyal Orion, Ramat - Efal (IL); Ronny Winshtein, Ramat - HaSharon (IL); Oren Goldshtein, Nahariya (IL)

(21) Appl. No.: 13/929,405

(22) Filed: Jun. 27, 2013

Related U.S. Application Data
(63) Continuation of application No. PCT/IL2011/050082, filed on Dec. 27, 2011.
(60) Provisional application No. 61/428,432, filed on Dec. 30, 2010.

(51) Int. Cl. A61B 5/0215; A61B 5/08; A61B 5/00

(52) U.S. Cl. CPC A61B 5/0215; A61B 5/6847; A61B 5/08
USPC 600/486; 600/300; 600/529

(57) ABSTRACT

The present invention relates to devices, systems and methods for delivering a sensory implant along a selected linear pierced path of introduction to a wall target located on an external surface of an internal body organ; and implanting the sensory implant in the wall of the internal body organ.

### RELATED APPLICATION

This application is related to U.S. Provisional Patent Application No. 61/428,432 filed Dec. 30, 2010 and entitled “PERCUTANEOUSLY IMPLANTABLE VASCULAR SENSOR”, the disclosure of which is incorporated herein by reference in their entirety, as if fully set forth herein.

### TECHNICAL FIELD

The present system, device and method relate to the field of monitoring conditions of a target organ in the human body, and more specifically to implantable sensing devices adapted for measuring pressure and/or other information generating from a target organ in the human body.

### BACKGROUND

Continuous monitoring of mammalian organ function and detection of adverse events in the organ in real time is an ever growing quest in modern medicine, having benefits in saving patients' lives, diminishing hospitalization time and reducing total healthcare related expenses. In circumstances where continuous accurate monitoring is required, designated sensory implants may be deployed adjacent to or inside the organ to be monitored. Common surgical interventions for delivery and implantation of sensory implants include utilization of existing natural orifices and vessels for intra-luminal delivery (i.e., through the lumen of such orifices and vessels) in which a resilient catheter and/or an endoscope are used to deliver the implant along an intra-luminal path, for example in a vascular or a gastrointestinal passage. In cases where the internal organ is adjacent to the skin surface and/or is positioned in a surgically accessible body chamber, such as the abdominal cavity, trans-dermal minimally-invasive approaches are used (for example, using laparoscopic ports and/or instrumentation).

One major condition that would greatly benefit from continuous real-time implant based monitoring is Congestive Heart Failure (CHF). CHF has emerged as a major public health problem affecting close to 7 million Europeans and 5 million North Americans each year. Despite new and more effective pharmacological and non-pharmacological therapeutic strategies, the prognosis of patients with CHF remains poor. Because of its progressive and unstable nature, many patients require multiple hospital admissions for CHF decompensation (a change in heart failure signs and symptoms resulting in a need for urgent therapy or hospitalization).

The growing cost of health care for heart failure patients is well documented. Although CHF patients are often affected by other significant co-morbidities, recurrent episodes of decompensation resulting from volume overload or changes in ventricular function are the most frequent cause for hospitalization in those patients.

Symptoms leading to hospitalization usually occur late in the course of decompensation and with current medical diagnostic methods, in some patients, decompensation requiring hospitalization may go unnoticed or not be attended to in due time in face of the lack of symptoms. The use of physical findings such as rales, edema or elevated jugular venous pressure as indicators for CHF patient condition evaluation has limited accuracy. Of the general measures that should be pursued in

patients with CHF, possibly the most effective yet least utilized measure is close and continuous monitoring of clinical signs as expressed by parameters of the cardiovascular system in general and specifically of the heart. Such continuous monitoring can prevent unnoticed clinical deterioration and late hospitalization.

One such parameter, among others, is left atrial pressure (LAP). CHF decompensation is characterized by increased LAP causing symptomatic pulmonary congestion and frank edema. Though possible precipitants of acute CHF are numerous, they all share the common precipitating factor of elevated LAP.

The rise in LAP usually is gradual and precedes symptom onset. Continuous hemodynamic monitoring of LAP may enable detection of increases in LAP before the onset of observed clinical deterioration.

In recent studies LAP estimates derived from right ventricular hemodynamics or thoracic impedance monitoring were used to guide treatment. The results of these studies suggest that there may be an important role for hemodynamic monitoring of CHF in selected outpatients. Placement of an LAP sensor in, for example, the left atrium would enable such monitoring by providing valuable data in real time regarding variations in LAP.

### SUMMARY OF THE INVENTION

The current invention seeks to provide a system, device and method for implanting a sensory implant in a body organ.

There is thus provided in accordance with the current system, device and method of the present invention a system including a sensory implant adapted to sense a change in at least one parameter such as, for example, pressure, associated with a condition and/or a performance of an internal body organ. The internal body organ may be left atrium. The internal body organ includes a wall that may include muscle tissue and/or be pulsatory.

In some exemplary embodiments, the system of the present invention includes an implant delivery device including a longitudinal body enclosing a lumen communicating with an open free end thereof and adapted to provide a contained passage, optionally of 1.5 mm or less in diameter, along a linear pierced path between an entry point and a wall target associated with the internal body organ. In some embodiments of the invention, the implant delivery device further comprises an introducer releasably connected to the sensory implant and adapted to advance the sensory implant from an enclosed position in the lumen to a protruded position distal to the free end. In some embodiments, the implant delivery device is operative to penetrate the wall from the exterior surface and to release the sensory implant in a selected location in the wall.

In some embodiments in accordance with the current system, device and method the introducer is coupled to the sensory implant by a threaded mechanism and optionally, when connected to the sensory implant, may allow electrical connectivity between the sensory implant and a remote device provided outside the body. The remote device may be including an electrical power source and/or a signal receiver. In some embodiments of the invention, the introducer includes a distally positioned coiled member provided in a direct electrical contact with a proximally positioned electrically conductive member, the coiled member is adapted for inductive coupling with an implant conductor provided with the sensory implant.

In some embodiments in accordance with the current system, device and method the system also includes a positioning verification analyzer communicative with the sensory implant prior to the releasing thereof and adapted to correlate between a signal transmitted by the sensory implant, including one or more of a sensing component, a signal processing component, a telemetry component, a stored identifying data and a micro electro mechanical system (e.g., a pressure transducer type, optionally a capacitive transducer).

In some embodiments, the point of entry may be percutaneous and/or adjacent an external surface of the wall. In some embodiments, the wall target is located on an external surface of the wall or is located in the wall.

In some embodiments of the invention, the linear pierced path may be straight and may optionally be at least 1 cm, optionally at least 5 cm, optionally at least 10 cm, optionally at least 20 cm in length, or higher or lower or intermediate.

In some embodiments, the sensory implant includes at least one retention member capable of altering from a longitudinal form, when confined to lumen boundaries, to an angled form when protruding out the lumen, optionally an angled form which is perpendicular to the longitudinal form. In some embodiments, the at least one retention member includes a resilient portion being at a higher stressed condition when the at least one retention member is at the longitudinal form than when at the angled form. In some embodiments, the at least one retention member is part of an expandable anchor configured to expand from a collapsed transverse size imposed by the lumen boundaries to an expanded transverse size being 2 mm or more greater than a maximal diameter of the free end.

In some embodiments, the sensory implant includes at least one retention member capable changing at least one of its spatial configuration and orientation so that to be capable of fitting inside the lumen. In some embodiments, the sensory implant includes at least one retention member capable of changing at least one of its spatial configuration and orientation from a first folded state inside the lumen to a second released state outside the lumen; and wherein the axis of the largest dimension in the folded state and the axis of the largest dimension in the released state are perpendicular to each other. In some embodiments, the sensory implant includes at least one retention member capable of changing its spatial orientation from a first folded state inside the lumen to a second released state outside the lumen; and wherein the longitudinal axis of the retention member in the folded state and the longitudinal axis of the retention member in the released state are perpendicular or at an angle to each other. In still some embodiments, the sensory implant includes at least one retention member capable of changing at least one of its spatial configuration and orientation from a first state inside the lumen folded along the longitudinal axis of the implant to a second outstretched state outside the lumen having an outstretched diameter along an axis perpendicular to the longitudinal axis of the implant and the outstretched diameter of the retention member is 2 times, optionally 3 times, optionally 5 times, optionally 10 times or more, the diameter of the sensory implant.

In an exemplary embodiment, the sensory implant includes at least two separate capsules, each housing and/or is connected to an electronic or electromechanical member, inter-connected by a retention member (e.g., a wire or a thin bar), wherein each capsule is capable of changing orientation from a first folded state inside the lumen to a second released state outside the lumen. Optionally, the longitudinal axes of the capsules in the folded state are parallel to the longitudinal axis of the retention portion; and optionally the their longitudinal axes in the released state is perpendicular or at an angle to the longitudinal axis of the retention portion.

In some embodiments of the present invention, the sensory implant includes an expandable inductor configured to expand from a collapsed transverse size imposed by the lumen boundaries to at least 3 mm, optionally to at least 5 mm, optionally to at least 10, optionally about 8 mm, in diameter, when is unstressed (e.g., when no compressive forces are applied thereto and/or where no external object confine it to smaller boundaries). In some embodiments, the expandable inductor is further configured to perform as an anchoring device when expanding over the collapsed transverse size. Optionally, the expandable inductor includes an induction coil interlaced with an antenna coil.

In another aspect of some embodiments, there is provided a sensory implant for implantation in a muscular wall of a cardiac left atrium adapted to sense pressure changes developing inside the atrium, the sensory implant includes: a capsule being 1.5 mm or less, optionally about 1 mm or less, in diameter, and containing a micro electro mechanical sensing component, wherein the capsule is provided coupled with an inductor and with an anchor adapted to alter from a longitudinal form, equal or less than the capsule diameter, to an angled form having a diameter greater than capsule diameter, optionally by at least 2 times, optionally by at least 3 times, optionally by at least 5 times, optionally by at least 10 times. In some embodiments, the anchor comprises the inductor and/or is interlaced thereto.

In another embodiment, the sensory implant is including a housing encapsulating passive electrical components including a micro electro mechanical sensing component, optionally being positioned at a distal portion, and a wireless transmission coil component, optionally being positioned at a proximal portion, along housing length, and including a retaining member adapted to prevent axial movement of the sensory implant in the muscular wall. In some embodiments, the wireless transmission coil and/or the retaining member are adapted to alter from a longitudinal form, equal or less than the housing diameter, to a surfaced form having a diameter at least 3 times greater than the housing diameter. Optionally, alternatively or additionally, the sensory implant includes a housing encapsulating passive electrical components including a micro electro mechanical sensing component positioned on one side/at one end thereof and a wireless transmission coil component positioned at another/at a different end thereof; wherein the housing is 1.5 mm or less in diameter and includes a retaining member adapted to prevent axial movement of the sensory implant in the muscular wall; and wherein the wireless transmission coil and/or the retaining member are adapted to alter from a longitudinal form, equal to or less than the housing diameter, to an outstretched form having a diameter at least 3 times greater than the housing diameter. In some embodiments, the retaining member includes the wireless transmission coil.

There is also provided in accordance with the current system, device and method of the present invention a method including introducing an implant delivery device carrying an implant along a selected linear pierced path and arriving at a wall target located on an external surface of a cardiovascular organ wall; and anchoring at least a portion of the implant in the cardiovascular organ wall. Optionally, the wall target includes the site of implantation.

In another embodiment there is provided another method including introducing an implant delivery device carrying an implant such as, for example, a sensory implant, optionally including a pressure sensor, along a selected linear pierced path and arriving at a wall target located on an external surface of an internal body organ; and anchoring at least a portion of the implant in the internal body organ wall. Optionally, the site of implantation includes or is included in the wall target. Optionally, the selected linear pierced path is selected prior to percutaneous introduction of the implant delivery device. In some embodiments, the method is further including implanting the implant in a site of implantation in the wall of the internal body organ.

In some embodiments, the selected linear pierced path is a direct path that accommodates introduction and advancement of the implant delivery device in a straight line. In some embodiment, the delivery device passes at least once through lung tissue during its advancement along the linear pierced path. In some embodiment, the method also includes verifying implant location prior to the anchoring. In some embodiments, the selected linear pierced path is selected prior to the introducing. Optionally, such selecting is followed by a preliminary imagery scan analysis. Optionally, the method also includes selecting an entry point defining the linear pierced path in-between the organ target.

In some embodiments of the invention, the implant delivery device comprises:
(a) A longitudinal body enclosing a lumen communicating with an open free end thereof and adapted to provide a contained passage along the linear pierced path, and
(b) An introducer releasably connected to the sensory implant and adapted to advance the sensory implant in the lumen; and
   wherein the method further including: advancing the introducer from an enclosed position in the lumen to a protruded position distal to the free end.

In another embodiment there is provided a method including selecting a wall target located on an external surface of an internal body organ, choosing a linear path between an entry point and the wall target, using the system described hereinabove to pierce across tissues along the linear path, penetrating the wall of the internal body organ from the external surface and the sensory implant in the wall. In some embodiments, the method further comprising: advancing the sensory implant from an enclosed position in the lumen to a protruded position distal to the free end.

In yet another embodiment the implant delivery device is included of a rigid elongated member having an open free end and a lumen extending at least partially along a length of the rigid elongated member, and wherein the lumen directly communicates with the open free end. In still another embodiment the implant delivery device is further included of a selectively attached implant. In another embodiment the percutaneous implant point of entry is identified based on the selected path of introduction. In yet another embodiment the method also includes verifying correct positioning of the delivery device tip and the implant carried thereby at a site of implantation at the target, deploying and implanting the implant at the site of implantation, verifying correct implantation of the implant and the functionality thereof and deploying an implant anchor and anchoring the implant at the site of implantation.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A, 1B and 1C are cross-section view simplified illustrations of exemplary locations in the cardiovascular system for placement or implantation of sensory implants, in accordance with exemplary embodiments of the present invention;

FIG. 2 is a plan view simplified illustration of an exemplary embodiment of an exemplary sensory implant deployed in an implant delivery device, in accordance with exemplary embodiments of the present invention;

FIGS. 3A and 3B are cross-section view simplified illustrations of exemplary deploying of a sensory implant by an introducer, in accordance with exemplary embodiments of the present invention;

FIGS. 4A and 4B are cross-section view simplified illustrations exemplary deploying of a sensory implant by a delivery device, in accordance with exemplary embodiments of the present invention;

FIGS. 5A, 5B and 5C are cross-section view simplified illustrations of packing configurations of different exemplary sensory implants inside delivery systems, in accordance with exemplary embodiments of the present invention;

FIG. 6 is a simplified cross-section view illustration of a human body at the level of the left atrium and an exemplary path of introduction for an implant delivery device, in accordance with exemplary embodiments of the present invention;

FIG. 7 is a flow chart illustrating an exemplary method of implanting a sensory implant in an organ wall, in accordance with exemplary embodiments of the present invention;

FIGS. 8A, 8B and 8C are a cross-section view simplified illustration of an exemplary sensory implant in different implanting schemes, in accordance with exemplary embodiments of the present invention;

FIG. 9 is a simplified illustration of an exemplary component electrical circuit of an exemplary implanted sensory implant, in accordance with exemplary embodiments of the present invention;

FIGS. 10A and 10B are a perspective and cross-section view simplified illustrations of an exemplary two-members type sensory implant, in accordance with exemplary embodiments of the present invention;

FIGS. 11A and 11B are perspective view simplified illustrations of an exemplary flower-type sensory implant, in accordance with exemplary embodiments of the present invention;

FIGS. 12A, 12B and 12C are perspective view and cross-section view simplified illustrations of an exemplary double-flower-type sensory implant, in accordance with exemplary embodiments of the present invention;

FIGS. 13A and 13B are perspective and cross-section view simplified illustrations of an exemplary hanging-type sensory implant, in accordance with exemplary embodiments of the present invention;

FIGS. 14A and 14B illustrate complete and transversally cut perspective views of an exemplary sensory implant releasably connectable to an introducer, in accordance with exemplary embodiments of the present invention;

FIGS. 15A and 15B illustrate perspective views of an implant delivery device and an introducer releasably connectable to the sensory implant of FIGS. 14A and 14B, in accordance with exemplary embodiments of the present invention;

FIGS. 16A and 16B illustrate perspective and cross-sectional views of the interconnected sensory implant of FIGS. 14A and 14B to the introducer of FIG. 15B in packed configuration, in accordance with exemplary embodiments of the present invention; and

FIGS. 17A, 17B and 17C illustrate perspective and cross-sectional views of deployment stages of the sensory implant of FIGS. 14A and 14B in a wall of an internal body organ, in accordance with exemplary embodiments of the present invention.

### DESCRIPTION OF EMBODIMENTS OF THE INVENTION

The following preferred embodiments may be described in the context of exemplary cardiovascular related sensory implants implantations for ease of description and understanding. However, the invention is not limited to the specifically described devices and methods, and may be adapted to various clinical applications without departing from the overall scope of the invention, for example implantations of sensory implants in other regions or internal organs of the body and/or implantations of other non-sensory implants (e.g., in a cardiovascular organ or in any other internal body organ.

It is to be understood that the terminology used herein is used for the purpose of describing particular embodiments only, and is not intended to limit the scope of the invention. Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

The term “patient” as used herein refers to a mammalian individual afflicted with or prone to a condition, disease or disorder as specified herein, and includes both humans and animals.

The term “sensory implant” as used herein refers to an artifact which includes a sensor or a sensing mechanism designed to receive a signal or stimulus and responds to it in a distinctive manner. The signal or stimulus can be a change in condition and/or a performance of an internal body organ (for example, a change in pressure, temperature, PH or others). The sensory implant may also include other means, optionally provided in an electrical circuit, designed to generate readable or measurable information corresponding to received signal or stimulus and/or designed to transmit a digital signal correlative to the change in condition and/or performance. The sensory implants of this invention may be considered “micro-” or “micro sized” in the sense they are limited in size, and more particularly characterized in a maximal diameter of 2 mm or less, and in some instances of 1.5 mm or less. The sensory implants may be of any length, usually depending optionally in the range of 1 to 20 mm, optionally in the range of 3 to 10 mm.

The term “Organ” or “body organ” as used herein refer to a collection of tissues joined in structural unit to serve a common function. The term “internal body organ” as used herein refers to organs, usually chambers or conduits in a patient's body enclosed with a wall, that are commonly positioned distally to a skin tissue and/or muscle tissue and/or bone tissue. Internal body organs may include, but are not limited to, the heart and chambers thereof, veins, arteries, brain, lung, kidney, muscles, ureter, bladder, urethra, mouth, esophagus, stomach, small and large intestines.

The term “wall” as used herein as in a “wall target” or “internal body organ wall” refers to the barrier of the internal body organ, either completely or only partially covering it, having a thickness, and comprising a soft tissue (connective and/or muscular). For example, a wall of a heart chamber will commonly include several layers of soft tissues, including: external fibrous layer, parietal pericardium, visceral pericardium, myocardium and endocardium. An “external surface” of a body organ refers to the overlying surface of the wall at the exterior of the internal body organ. A “wall target” as used herein refers to an area or a point adjacent, on or in the wall or the external surface, to which a sensory implant is carried prior to or as part of deployment and implantation in the wall at the site of implantation. The term “site of implantation” as used herein refers to the physical location where an implant, such as a sensory implant, is inserted into a wall of a body organ and fixedly anchored thereto.

The term “contained passage” as used herein refers to a man-made, usually elongated, conduit, lumen or track through which the sensory implant can be advanced or delivered to a target point, for example a wall target. The contained passage can be a lumen enclosed with a rigid or semi-rigid protective covering, as for example a needle lumen. The term “entry point” as used herein refers to a point distal to the wall target through which the sensory implant, the contained passage and/or the implant delivery device is initially inserted. The entry point may be located on patient's skin (therefore considered a percutaneous entry point) or at an internal tissue.

The terms “path” or “path of introduction” as used herein refers to a chosen course or route along which the sensory implant, the contained passage and/or the implant delivery device travels. The term “linear pierced path” as used herein refers to a continuous non-natural path in the human body between an entry point and a target point, for example a wall target, that crosses through tissues and/or organs in patient's body. For example, a liner pierced path can be made by percutaneously inserting a sharpened needle in patient's body and advancing it towards a wall target. The linear pierced path excludes naturally existing lumens and conduits such as of the vascular or gastrointestinal systems.

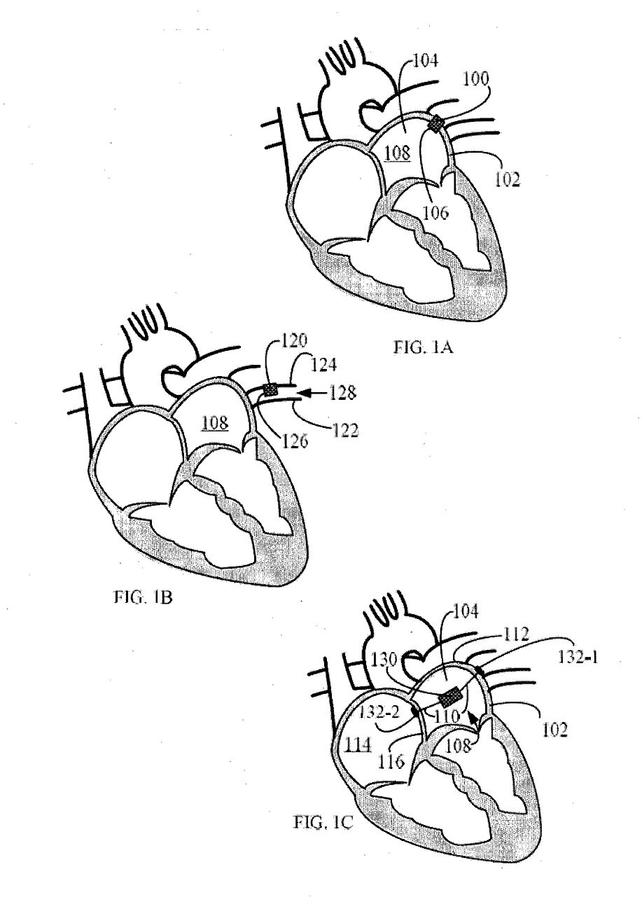

Reference is now made to FIGS. 1A, 1B and 1C are cross-section view simplified illustrations of exemplary locations in the cardiovascular system for placement or implantation of sensory implants, in accordance with exemplary embodiments of the present invention. As shown in FIG. 1A, a sensory implant 100 may be implanted in a wall 102 of the left atrium 104. Sensor 100 may be fully implanted within wall 102 or be partially implanted in wall 102 and have a portion 106 partially protruding into atrial lumen 108.

In FIG. 1B, a sensory implant 120 may be implanted in a wall 122 of a pulmonary vein 124 in propinquity to the pulmonary vein-left atrium junction. Sensor 120 may be fully or partially implanted in wall 122 and have a portion 126 partially protruding into the lumen 128 of pulmonary vein 124. Alternatively and optionally, sensor 120 may be partially implanted in wall 122 of vein 124 having a portion (not shown) protruding outwardly outside the external surface of vein 124 wall 122. Alternatively and optionally, sensor 120 may be coupled to pulmonary vein 124 employing a bracing device or a strip (not shown) placed along the circumference of vein 124.

FIG. 1C illustrates a third example of a location for implanting a sensory implant. In FIG. 10 sensor 130 is suspended by a wire 110 in lumen 108 of left atrium 104 from two or more anchors 132 fully or partially implanted in wall 102 of atrium 104. Alternatively and optionally, anchor 132-1 may abut the external surface 112 of left atrium 104 wall 102 whereas anchor 132-2 may abut the right atrium 114 side of atrial septum 116.

In some embodiments, any one of sensory implants 100, 120 and 130 may be operative to measure one or more hemodynamic parameters selected from a group of parameters including blood pressure, blood velocity, degree of blood oxygenation (i.e., blood oxygen saturation) and temperature within the lumen of the monitored target organ. Additionally and optionally, sensory implants 100, 120 and 130 may be operative to measure one or more of existence and location of cyclic and/or dynamic blood flow and presence of continuous turbulent flow across the sensor boundaries.

A sensory implant if implanted inside the wall of an organ may be operative to measure one or more parameters associated with the physiological state of the organ such as wall myocardial contractility (e.g., in a wall of a heart chamber), wall tissue pulsation, contraction and wall tension. Measured parameters regarding muscle contraction forces may be used to derive information indicative of one or more changes in a subject's condition in general and especially changes that may be indicative of the subject entering into an acute condition.

In some embodiments, a plurality of sensory implants may be implanted in two or more locations such as, for example, two or more heart chambers or one or more heart chambers and a major blood vessel. For example, a plurality of sensory implants, one of which may be located in the left atrium and a second in the Vena Cava, may provide data regarding various parameters (e.g., pressure and/or saturation) both in the pulmonary (lung) circulation system and in the systemic venous circulation system such as filling pressures of the right atrium and volume status of the hepatic, intestine and lower limbs veins; In some embodiments, a plurality of sensory implants may be applied to monitor filling pressures of both the left and the right atria optionally collecting data for managing a subject's volume status and/or reducing exacerbations rates. Two or more sensory implants, operative to measure the same parameter may be implanted in two or more organs in propinquity to and communicating with each other for improved overall measurement accuracy. Such an example may be implanting one or more sensory implants such as sensory implant 100 in wall 102 of left atrium 108 and one or more sensory implants such as sensory implant 120 in wall 122 of vein 124 (not shown).

Organs to be monitored and location of sites of implantation may be determined in accordance with the associated clinical application, as shown in the examples hereinafter. (1) Selecting the right atrium, superior or inferior vena cava as sites of implantation may enable to monitor right sided or combined left and right sides heart failure to better manage HF patients and reduce fluid retention and its complications. (2) Selecting the pulmonary arteries as sites of implantation may enable monitoring primary pulmonary hypertension, help titrate treatment medication and assist pharmaceutical companies to develop and test new drugs. (3) In cases of aortic aneurysms, selecting the aneurysm sac as a sensory implant site of implantation, for example, following implantation of a stent graft to repair the aneurysm, may enable close follow up of the patient to evaluate if there is an endo leak from the aorta to the aneurysm sac. This may replace a CT angiogram, which today is the method of choice to assess the sac. An implanted sensory implant may detect endo-leaks earlier and reduce radiation dose. (4) Implanting sensory implants in the right and left atrium and ventricles may also enable mapping the pressure gradients across the various heart valves and the blood pressure in each of the various heart chambers and enable optimization of the synchronization between the different heart chambers and their contractility by, for example, optimizing a pacemaker performance. In this case, one or more sensory implants may be implanted in walls of different heart chambers, as desired.

Sites of implantation of the present invention are not limited to cardiovascular system organs and one or more sensory implants may be implanted at other sites, including in walls of organs such as the intestinal tract, the gall bladder, the urinary bladder, the brain ventricles (to monitor hydrocephalus treatment), the subarachnoid space and others. In some embodiments, the sensory implant is adapted for long-term coupling to or implantation in an organ wall employing designated anchoring means as will be described in detail hereinafter.

The inventors have developed a system, devices and method for delivering, positioning and deploying or implanting a sensory implant in a body organ by first introducing a small-diameter implant delivery device as described hereinafter. In one aspect of exemplary non-limiting embodiments, some systems, devices and methods are designed to be implemented by employing minimally invasive procedures while minimizing undesired injury to body tissues or by directly visually targeting a body organ during invasive surgical procedures such as, for example, open heart surgery.

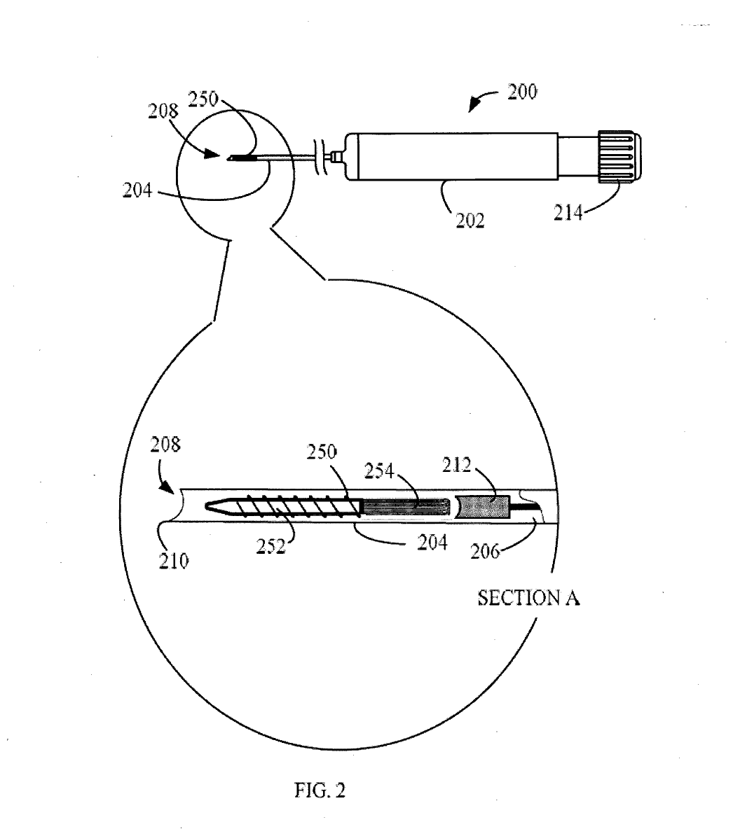

Reference is now made to FIG. 2, which is a plan view simplified illustration of an exemplary embodiment of a system 200 comprising a sensory implant 250, which includes an implant body 252 coupled with an anchor 254, and an implant delivery device 202, in accordance with the current system, device and method. Delivery device 202 may include a handle 214 and a distally projecting longitudinal body 204 enclosing a lumen 206 extending at least partially along a length of longitudinal body 204 and communicating with an open, sharp distal free end 208. Alternatively and optionally, distal free end 208 may temporarily house a trocar or a mandrel (not shown) facilitating advancement of longitudinal body 204 through tissue while avoiding bending or buckling and/or entrapping tissue in lumen 206. Longitudinal body 204 may be in a form of a needle having a diameter less than 2 mm. In some embodiments the diameter of longitudinal body 204 may be between 0.5 and 2 mm, optionally 1.0 and 1.5 mm, and its length may be between 10-25 cm, optionally 12-23 cm, optionally 15-20 cm.

Elongated body or needle 204 may be sufficiently firm so that a force applied to axially advance longitudinal body 204 through tissue will be maintained along the projection of its longitudinal axis and be sufficient to enable distal free end 208 to penetrate tissue. Additionally, a non-bending longitudinal body 204 may be easily manipulated and provide high accuracy in aiming distal free end 208 and steering it to a specific point on a tissue wall via a chosen path of introduction. Longitudinal body 204 may also be operative to releasably accommodate sensory implant 250, as will be described in greater detail below.

In FIG. 2, enlarged section A is a sectional view of longitudinal body 204 in which a portion has been removed for illustration purposes revealing sensory implant 250 enclosed in lumen 206 of body 204. Sensory implant 250 may be packed in a folded or closed configuration so that to have a diameter equal or smaller than of lumen 206 accommodating thereof, and be positioned and/or shaped along the longitudinal axis of lumen 206 so that to retain the ability to slidingly and axially move along lumen 206, as will be further described in greater detail below.

Free end 208 may be operative to penetrate the exterior of an organ wall and deploy sensory implant 250 in a selected location in a wall of an organ. Free end 208 may also include a beveled and/or sharp tip 210 operative to cut through tissue. In some embodiments, sensory implant 250 is a micro sized implant, and for example may have a diameter between 0.1 to 2 mm, optionally 0.5 to 1 mm; optionally 1 to 20 mm, optionally 5 to 10 mm in length.

A pusher type introducer 212 may be operative to advance axially through lumen 206 of system elongated body or needle 204 by manual activation of actuator 214 on handle 202 or by employing a motor. Sensory implant 250 and introducer 212 are sized and configured for axial travel in lumen 206, including a forward travel to a point at which introducer 212 and/or sensory implant 250 protrudes a selected distance from the shaft's distal free end. Optionally, introducer 212 may be operative to rotate (not shown) while advancing through lumen 206. Alternatively and optionally, walls of lumen 206 may include a spiral groove corresponding to a matching thread on sensory implant 250 so that to apply rotational motion thereto about its longitudinal axis as it advances through lumen 206. Introducer 212 may be operative to advance through lumen 206 of body or needle 204 pushing a sensory implant 250 located ahead of pusher 212 through lumen 206. In such a configuration, neither pusher 212 nor sensory implant 250 includes means connecting therebetween.

Longitudinal body 204 may also be operative to enable introduction of fluids, including saline, medications, vitamins, tissue glue or other substances, either via lumen 206 or via a dedicated separate lumen (not shown). Additionally and optionally, delivery system 200 may also include a mandrel to prevent tissue from entering the needle during advancement.

Additionally and optionally, longitudinal body 204 may also include an inflatable balloon (not shown) in propinquity to free end 208 operative to deflect tissue away from the wall target. The inflatable balloon may be inflated and/or deflated manually or automatically employing filling media such as saline via a dedicated separate lumen (not shown).

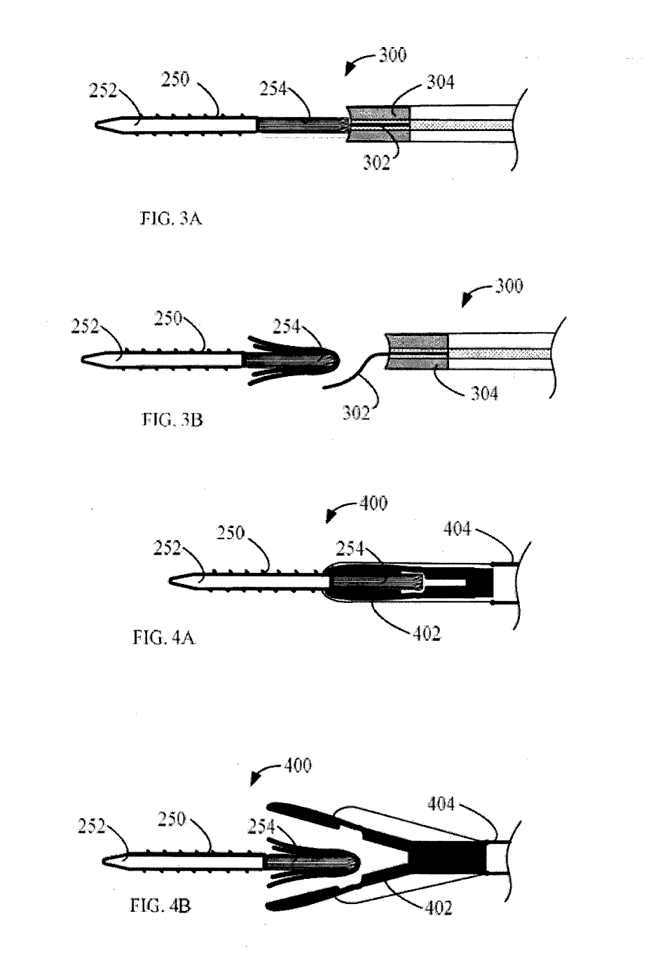

Reference is now made to FIGS. 3A and 3B are cross-section view simplified illustrations of exemplary deploying of sensory implant 250 by an introducer 304, as part of a system 300, in accordance with exemplary embodiments of the present invention system 300 also includes a longitudinal body having a lumen sized for enclosing sensory implant 250 and/or introducer 304, not shown in the figures, for ease of description and illustration only. In some embodiments of the invention, sensory implant 250 may include a body 252, anchor 254 and means for releasably connecting to a distal end of introducer 304, for example a threaded male/female mechanism (not shown) in sensory implant 250 for threading to a corresponding female/male member (not shown) provided in introducer 304 and/or at least one anchor configured for anchoring to lumen periphery not shown). In some embodiments, the sensory implant is selectively releasably connected to introducer 304.

Alternatively and optionally, sensory implant 250 may form a detachable tip of system 300 and be releasably secured thereto by a tether 302 secured to introducer 304. After implantation and as seen in FIG. 3B, tether 302 may be broken therefore releasing sensory implant 250 from system 300. After being released and removed from enclosing system 300, anchor 252 may expand to an expanded form characterized in enlarged transverse size (e.g., diameter), as will be further described in detail hereinafter.

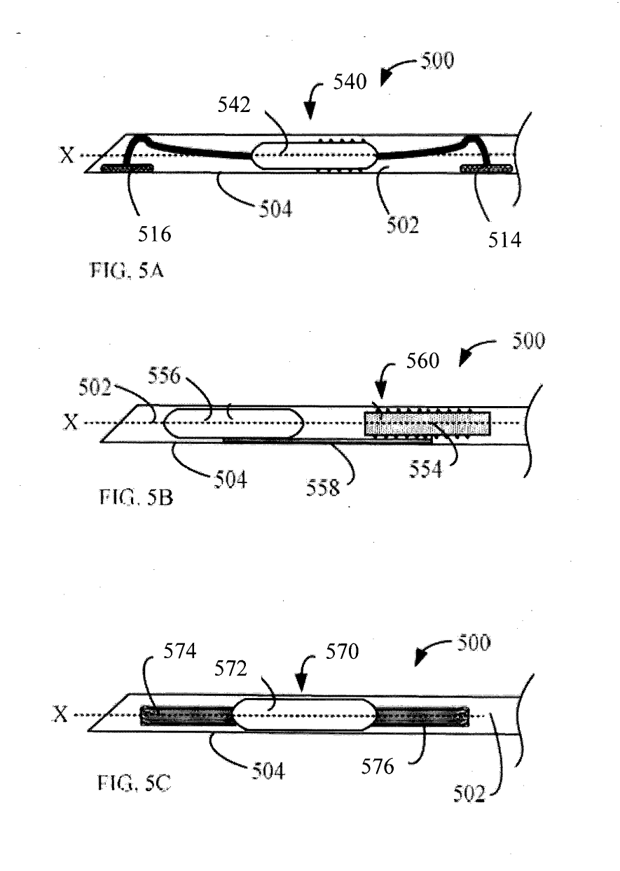

Reference is now made to FIGS. 4A and 4B are cross-section view simplified illustrations exemplary deploying of sensory implant 250 by a delivery device 400, in accordance with exemplary embodiments of the present invention. Sensory implant 250 may be releasably connected by a gripping mechanism 402 at the tip of a longitudinal body 404. Gripping mechanism 402 may be operative to firmly grip sensory implant 250 (FIG. 4A) and release its grip (FIG. 4B) once the device has been implanted. After being released, and as shown in FIG. 4B, anchor 252 may begin to expand towards implantation, as will be further described in detail below. Manipulation of gripping mechanism 402 may be manual employing an actuator (such as handle 214 previously shown in FIG. 2) or remote via a cable (not shown) or any other appropriate mechanism.

Reference is now made to FIGS. 5A, 5B and 5C which are cross-section view simplified illustrations of packing configurations of different exemplary sensory implants 540, 560 and 570, inside a lumen 502 of an elongated body 504, as part of system 500, in accordance with exemplary embodiments of the present invention. The very small diameter of lumen 502 necessitates a confined size for the sensory implant thereinside so that to retain the ability to slidingly and axially move along lumen 502.

Sensory implants 540 and 570 include one or more anchors (anchors 514 and 516 and anchors 574 and 576, respectively) operative to be deployed at both opposing sides of an organ wall to anchor the sensory implant thereto, as will be further described in greater detail below. In an alternative embodiment, and as seen in FIG. 5B, sensory implant 560 includes a first member or capsule 554 and a second member or capsule 556 inter-connected by a resilient or rigid wire 558. In such a way, the two capsules may also serve as anchoring members sandwiching therebetween a wall portion.

The anchors, 514, 516, 574, 576, for example, when deployed, may be capable of changing their spatial configuration from a folded configuration to an expanded configuration having a diameter along an axis perpendicular to the longitudinal axis of lumen 500. The longitudinal axis of any of capsules 554 and 556 (as shown in FIG. 5B), when deployed at the site of implantation, may be perpendicular or close perpendicular to the longitudinal axis retaining portion 558.

Referring back to FIG. 5A, sensory implant 540, at its deployed configuration (not shown) may occupy a volume having dimensions all of which greater than the diameter of lumen 502. Alternatively and optionally, sensory implant 540, provided with opposing anchors 514 and 516, may be capable of only changing orientation upon deployment by close to 90 degrees from a packed orientation parallel to longitudinal axis X of lumen 502 to a deployed orientation perpendicular or close to perpendicular to longitudinal axis X.

The constraints imposed by the dimensions (i.e., diameter) of lumen 502 necessitate a change in the spatial configuration and/or orientation of sensory implant 540 from an expanded or open configuration to a closed or folded configuration so that it fits inside lumen 502. As seen in FIG. 5A, this change may include arranging implant body 542 along the longitudinal axis X and in parallel to anchors 514 and 516. Alternatively, and as seen in FIG. 5B, capsules 554 and 556 of sensory implant 560 may be arranged along the longitudinal axis X and in parallel to wire 558. Alternatively, and as shown in FIG. 5C, as well as in FIGS. 2, 3A and 4A, this change may include folding of an anchor so that it may be capable of assuming an elongated form arranged, along the longitudinal axis X.

When in the packed configuration, the length of sensory implant 540, with or without anchors 514 and 516 becomes the largest dimension of thereof in diameter, for example double or more, optionally 5 times or more, optionally 10 times or more, larger than the largest dimension of its pre-deployed formation, i.e., in the packed or folded configuration.

Any one of the component of the systems and devices described above may be made by any applicable biocompatible material, such as metal alloys or polymers and may include radiolucent and/or radio-opaque portions and/or elements operative to change their geometry (i.e., expand, inflate and similar so that its advancement along the path of introduction may be monitored by appropriate imaging devices such as a CT or MRI scanner. Additionally and optionally, longitudinal body 200 may include on the surface thereof colored or etched markings to allow visual observation and evaluation of the depth of penetration into tissue such as would be done in, for example, open heart surgery.

In some embodiments, any of the systems described above may include safety mechanisms such as a securing tether element connected to the sensory implant thereby preventing untimely detachment from delivery needle. After implantation and verifying fixation, the tether may be detached and pulled out through the lumen.

In some embodiments of the system, method and device, deployment begins by first penetrating through, fully or partially, the wall of the internal organ from its external site towards its inner layers and/or surface, optionally using the sharp distal tip of the shaft, until reaching a maximal progression, which is the point at which the sensory implant maximally protrudes out of the shaft distal free end. Maximal progression may be achieved for example, by pushing the implant forward with respect to the shaft lumen using a pusher or an introducer and/or releasing, disconnecting or anchoring the implant to the organ wall. Alternatively and optionally, the sensory implant may be deployed by withdrawing the shaft while maintaining the sensory implant in-place, for example by using a pusher or introducer releasably connected or unattached to the implant.

In an exemplary embodiment, the sensory implant may include at least one distal anchoring or retaining member for resisting backward motion of the sensory implant, operative to be selectively or inherently deployed after the sensory implant protrudes out of shaft lumen at a first predetermined length.

Optionally, alternatively or additionally, the sensory implant may include one or more proximal anchoring or retaining member for resisting forward motion of the sensory implant, operative to be selectively or inherently deployed after the sensory implant protrudes out of shaft lumen at a second predetermined length greater than the first predetermined length. Once both distal and proximal anchors are deployed, the sensory implant may be released and/or disconnected from the delivery device. Before, during or after such release/disconnecting, other steps may be performed, optionally including at least one of: powering the sensory implant; testing, adjusting and/or calibrating subunits of the sensory implant; deploying a second sensor at a different implantation site; and/or collapsing at least one anchor and retracting the sensory implant back into shaft lumen for redeployment or discarding.

In some embodiments and as will be described in greater detail below, the use of systems and method of the present invention may enable at least one of: simplified and safe fixation, minimal protrusion (about 1-1.5 mm) of the sensory implant in the organ interior, repeatable fixation of the sensory implant, in the same position relatively to the atrial wall and its lumen, minimal movement of the sensory implant, minimal damage to the atrial pulsating wall (contracts and relaxes), conformability to the atrial wall, long term durability and minimal interference with the electrical activity on the atrial wall.

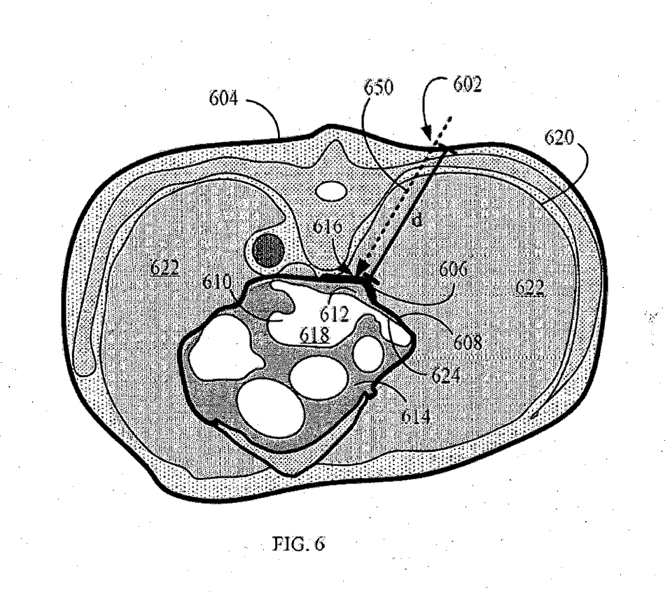

Reference is now made to FIG. 6 which is a simplified cross-section view illustration of a human body at the level of the left atrium and an exemplary path of introduction for an implant delivery device, in accordance with exemplary embodiments of the present invention. As described above, an implant delivery device, such as implant delivery device 202 (of FIG. 2) carrying a sensory implant, such as anyone of sensory implants 250, 540, 560 or 570, is percutaneously introduced through a selected entry point 602 on skin 604 along a selected linear pierced path marked by a broken arrow designated reference numeral 650, arriving at a wall target 606 located, for example, on an external surface 608 of the organ 610 wall 612, shown herein by virtue of example only for purposes of illustration may be the muscular wall of the heart 614 left atrium 610. Once at the target site of implantation 616, the implantable sensing device is deployed and implanted from external surface 608 of the organ 610 wall 612 towards the organ lumen 618 as will be described in greater detail below. The linear pierced path may be any chosen straight/direct linear path, a curvilinear or other route changing path, or any combination thereof. In an exemplary embodiment, the linear pierced path may be made using a percutaneous para-vertebral introduction approach.

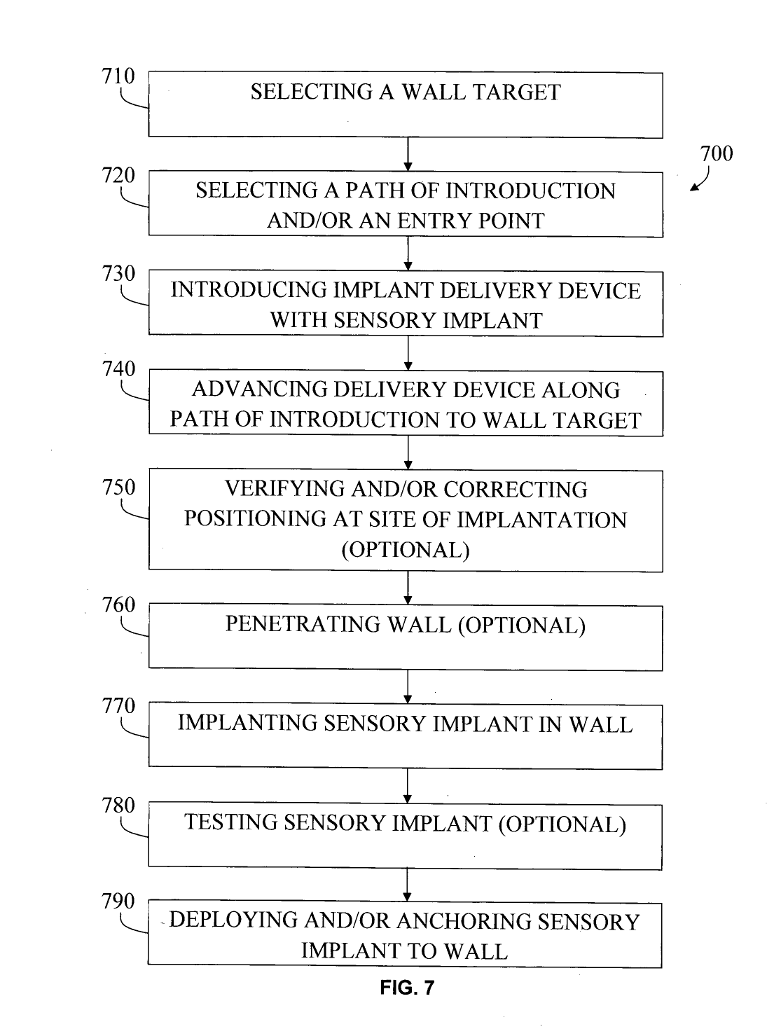

Reference is now made to FIG. 7, which is a flow chart illustrating a general exemplary method 700 of implanting a sensory implant in an organ wall, in accordance with exemplary embodiments of the present invention. As a first preliminary step, a wall target 606 is selected 710, optionally non-invasively, for example by employing imaging techniques such as CT or MRI scan of the external surface 608 of a wall of an organ 610 to be monitored. A path of introduction 650 is then selected 720 based on considerations such as a preferable entry point, distance (d) to the target, body organs detectable along the path, and, size of wall target, and possible other considerations.

In some embodiments, the site of implantation 616 encompasses wall target 606 or is included within wall target 606 or, alternatively, is adjacent to wall target 606. A preferable selected path of introduction 650 may be the shortest [i.e., smallest possible value of (d)] direct path possible, that accommodates introduction and advancement of a needle type delivery device in a straight line with minimal overall contact with body tissues or with minimal overall undesired injury to body tissues located along a linear pierced path. The path may be curvilinear or direct/straight linear extending from entry point 602 to target 606.

Alternatively and optionally, delivery and deployment of the sensory implant may be performed directly into wall 612 of body organ 610 obviating the need for a percutaneous path of introduction 650. Such would be the case in open heart surgery. A percutaneous entry point 602 may be identified based on the selected target 606 and a desired path of introduction 650. An implant delivery device similar to device 202 of FIG. 2 may be percutaneously introduced 730 and advanced 740 along the selected path of introduction 650 in a safe fashion driving and steering the rigid needle, for example in a straight line, thereby creating a linear pierced path, at least in most parts along the path of introduction 650, between entry point 602 and wall target 606. During its advancement 740 along path of introduction 650, the delivery device may pass at least once through, for example, at least one of the following tissue types, optionally in the following order: skin 604 (cutaneous, subcutaneous), pleura 620 and lung tissue 622.

As described above, the delivery device may include radiolucent and/or radio-opaque portions and/or elements operative to change their geometry (i.e., expand, inflate and similar so that its advancement along the path of introduction may be monitored by appropriate imaging devices such as a CT or MRI scanner.

Once the delivery device has arrived at wall target 606 on external surface 608 of left atrial wall 612 (FIG. 6), device positioning and/or alignment with respect to the wall 612 surface 608 may be verified and/or corrected 750 as necessary. Following such optional verification 750, the implant may be implanted 770 in the organ wall 612 at site of implantation 616, optionally by first fully or partially penetrating 760 through wall 612 with a sharp distal end from the external surface 608 towards luminal surface 624, and then deploying 790 the anchoring means provided with the sensory implant, such as, for example, anchor 252 to fixate to wall 612 in the selected position or angle of penetration.

The correct implantation of the sensory implant and the functionality thereof may be verified by testing 780 the sensory implant in-situ (e.g., by taking measurements of pressure and analyzing and/or correlating the data with known or stored information/database). Such testing 780 may follow or be followed by anchoring 790 the sensory implant in place. The exact location of the site of implantation and the implantation process end point may be verified by appropriate imaging devices and/or by readings picked up by a sensor in the sensory implant.

In some embodiment, for example, verification 750 and/or testing 780 of a sensory implant location and function may be carried out electrically. For example, the needle may also include one or more electrodes (not shown) in propinquity to free end 208 (FIG. 2) to measure impedance thus providing information by varying impedance regarding the location of the needle free end 208.

In some embodiments of the invention, once the delivery device has reached the wall target, a positioning and/or orientation verification may be optionally performed, for example if necessary or as per a protocol, either by using the sensory implant, by using another dedicated sensory means, visually by observing markers on the exterior surface of the delivery device needle and/or by using non-invasive imaging. After optionally performing the verification, deployment of the sensory implant in the implantation site can take place.

In some embodiments, the site of implantation encompasses the wall target or is included within the wall target, or alternatively is adjacent to the target so that the practitioner repositions and/or realigns the delivery device or any of its parts or members (e.g., the elongated slender body or a part thereof), either as part of the positioning and/or orientation verification or at a following step.

An exemplary method of implanting a sensory implant in the left atrium may include any or all of the following steps: (1) The subject is positioned (not shown) in a prone or prone oblique position; (2) A CT scan is performed to locate and identify a target on the wall of the left atrium; (3) The CT scan is also employed to plan the exact path of introduction and identify an exact cutaneous entry point and inter-costal space at which the delivery system needle will be inserted into the chest; (4) A needle, such as for example needle 200 (FIG. 2), optionally 12-26 Gauge in size, carrying a sensory implant is introduced into a subject's body with a mandrel in order to prevent tissue to enter the needle while advancing towards the left atrium; (5) Arrival at the external surface of the left atrium wall; (7) Verification of the location of the delivery needle and position of the sensory implant, employing, for example CT/MRI; (8) Delivery of the sensory implant to the site of implantation by penetrating the left atrium wall (102, FIGS. 1A and 1C) from the external surface (112, FIG. 1C) towards the atrial lumen (108, FIGS. 1A-1C) with the delivery system needle; (9) Verification of the needle tip location.

In some embodiments of the invention, once the needle tip location in the left atrium wall is verified, the delivery system needle may be employed to implant the sensory implant in the atrium wall. The implantation may be carried out employing an introducer 210 in a needle of the type shown in FIG. 2 or, alternatively, by releasing the grip of gripping mechanism 302 in a needle of the type shown in FIG. 3.

Verification of the needle tip location and implant functionality may be carried out employing any one or combination of the following methods: (1) CT/MRI with or without a contrast agent; (2) Readings that enable the physician to extract therefrom a left atrium pressure curve from a pressure sensor at the tip of the delivery device/needle or from the sensory implant itself; (3) Pulsating movement of the delivery device/needle corresponding to the heart rate; and (4) Oxygenated blood detected in an injector attached to the needle.

When implanting some types of devices, such as for example, a sensory implant comprising separated members or capsules, the longitudinal body of the delivery device may need to be slightly retracted back into the atrial wall before implantation of the device in the atrial wall.

Once in-place, the sensory implant may be attached and/or anchored to the atrial wall as described above. Delivery and deployment of a sensory implant may be performed directly into a wall of a body organ such as the left atrium obviating the need for a percutaneous path of introduction, such as in open heart surgery.

An exemplary method of sensory implant placement in the left atrium may include any or all of the following steps: (1) During the course of an open-heart procedure a target on the left atrium wall is identified visually; (2) A needle, such as for example needle 200 (FIG. 2), is introduced into a subject's thoracic cavity towards the target on the wall of the left atrium; (3) Arriving at the external surface of the left atrium wall; (4) Visually verifying the location of the delivery needle; (5) Penetrating the left atrium wall (102, FIGS. 1A and 1C) from the external surface (112, FIG. 10) towards the atrial lumen (108, FIGS. 1A-1C) with the delivery system needle.

The location of the needle tip may be verified by any one or combination of the following methods: (1) Readings that enable the physician to extract therefrom a left atrium pressure curve from a pressure sensor at the tip of the delivery device/needle or from the sensory implant itself; (2) Pulsating movement of the delivery device/needle corresponding to the heart rate; and (3) Oxygenated blood detected in an injector attached to the needle.

Once the needle tip location in the left atrium wall is verified, the delivery system needle may be employed to implant the sensory implant in the atrium wall. The implantation may be carried out employing an introducer 210 in a needle of the type shown in FIG. 2 or, alternatively, by releasing the grip of gripping mechanism 302 in a needle of the type shown in FIG. 3. Once in-place, the sensory implant may be attached and/or anchored to the atrial wall as described above.

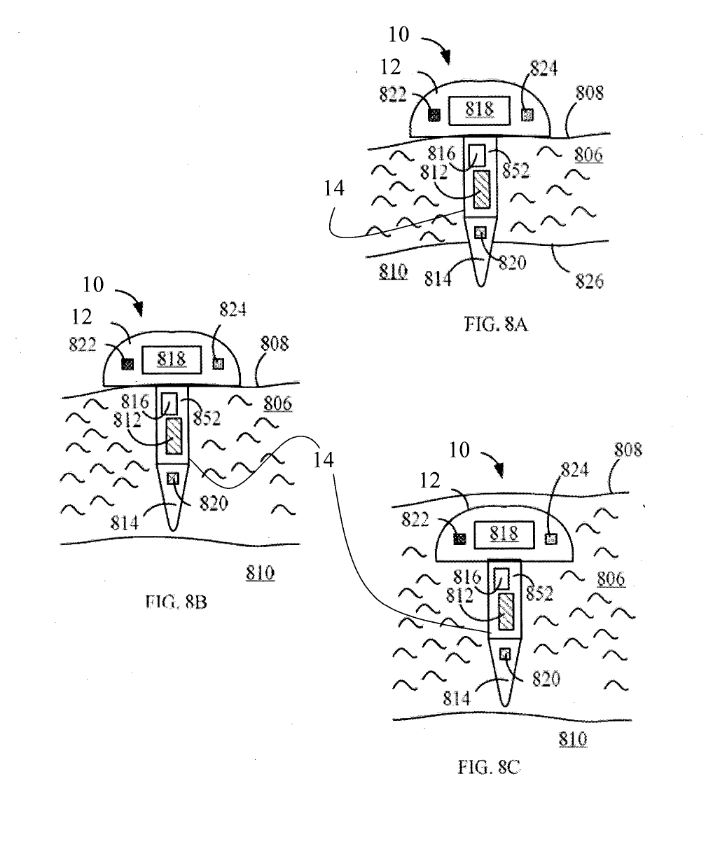

Reference is now made to FIGS. 8A, 8B and 8C which are a cross-section view simplified illustration of an exemplary sensory implant 10 in different implanting schemes, in accordance with exemplary embodiments of the present invention. Sensory implant 10 includes an implanted portion or body 14 and an anchoring portion 12. When implanted, portion 14 may penetrate a wall thickness 806 such as, for example, a wall of a body organ from the external surface 808 thereof. As shown in FIG. 8A, portion 14 may completely penetrate through tissue layer 806 and protrude into a lumen 810 defined by wall thickness 806 along a distance of at 2 mm, optionally at least 4 mm, optionally in the range of 3 to 10 mm, from internal surface 826 of wall thickness 806. Alternatively and optionally, sensory implant 10 may only partially or incompletely penetrate wall thickness 806 leaving a portion of wall thickness 806 un-penetrated as shown in FIG. 8B or be fully implanted inside wall thickness 806 as shown in FIG. 8C.

Either one or both of implanted portion 14 and anchor portion 12 may include, house or be one or more electrically driven components of sensory implant 10 including an electrical rechargeable power storing component 812, a sensing component 814, a signal processing component 816, an induction coil for receiving inductive energy (not shown) and a telemetry component 818. Sensing component 814 may be capable of transforming a change in physiological conditions associated with a performance or a condition of the monitored internal organ to a mechanical, electrical or chemical signal. In some embodiments, sensing component 814 may include at least one sensor operative for continuous measurements at relatively low power consumptions. In some embodiments, the sensor is micro or nano sized, for example a micro mechanical based sensor such as a micro electro mechanical system (MEMS), a nano electro mechanical system (NEMS), a micro optic mechanical system (MOMS), a micro optical electro mechanical system (MOEMS), a biological micro electro mechanical system (BioMEMS), a “lab-on-a-chip” system (LOC), or any combination thereof.

Rechargeable power storing component 812 may include a power conditioning circuitry mechanism that detects the level of power delivered and switches on the other components when adequate power levels are reached. The power conditioning circuitry mechanism may employ application-specific integrated circuitry (ASIC) and be included in signal processing component 816.

In some embodiments, signal processing component 816 may digitize the readouts from sensing component 814, process the readouts and prepare them for transmission. Processing may include a unique digital identification (signature) for identification and authentication of device 250 and the subject being monitored. The digital identification may be transmitted to an external unit by telemetry component 818 as will be explained in detail below. Signal processing component 816 may be an application specific integrated chip (ASIC). The ASIC may be encapsulated within a seamless biocompatible and flexible sheathing, such as silicon, and easily integrated with the additional components employing MEMS technique.

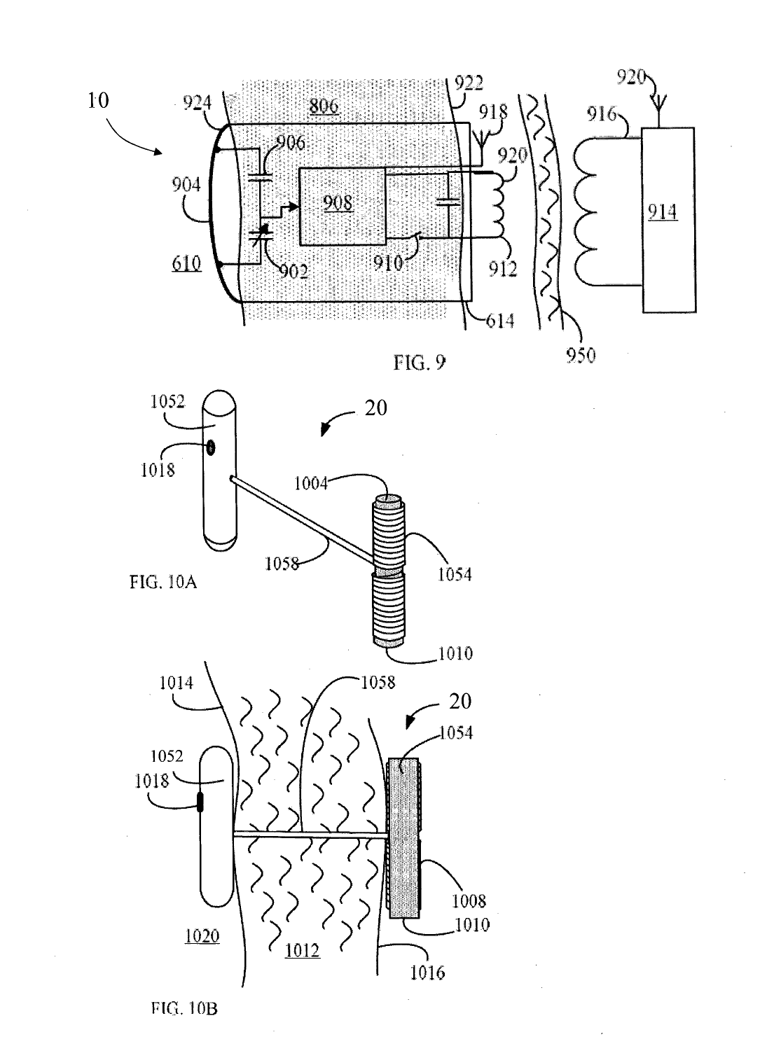

FIG. 9 is a simplified illustration of an exemplary schematic electrical circuit of sensory implant 10 in accordance with the current system, device and method. Sensing component 814 implanted fully or partially in a tissue layer 806 may include, for example, a MEMS capacitive pressure transducer 902 and an external unit 914 outside body wall 950.

By way of example only, a MEMS capacitive pressure transducer 902 may be in contact with a membrane 904 wherein changes in the pressure effected on membrane 904 correspondingly vary the capacitance of transducer 902 in comparison to a reference fixed capacitor 906. The detected differential capacitance between capacitor 906 and capacitive transducer 902 is converted by converter 908 into a signal such as electronic module output pulses. The sampling data may be expressed in the pulses frequency. Alternatively an optionally, the electronic module output pulses may, for example activate an on/off switch 910 in which case the sampling data may be expressed in the on and off durations of the signal.

The sampling data may be expressed, for example via switch 910, by variations in the impedance of a load 912 having an impedance that matches that of a load 916 of external unit 914. External unit 914 may, in turn, be operative to reconstruct the sampled data from the detected changes in the impedance of load 912. In this configuration the electrical circuitry of sensory implant 10 may employ magnetic inductance (implemented as a transformer) between external unit 914 and sensing component 814 for remote power feeding to charge electrical rechargeable power storing component 812 (FIG. 8). The inductance coil 920 may also be employed as part of telemetry component 818 to communicate with external unit 914 outside the body wall.

Alternatively and optionally, the differential capacitive signal may be modulated and transmitted to external unit 914 via a separate antenna 918 and received by an antenna 920 on external unit 914. Additionally or alternatively, antenna 918 may also be employed as part of telemetry component 818 and receive transmissions from antenna 920 of external unit 914. In this configuration magnetic inductance (implemented as a transformer) may be used only for remote power feeding to charge electrical rechargeable power storing component 812 (FIG. 8).

Either one or both of inductance coil 920 and antenna 918 may be placed outside the external surface 922 of wall 806, while the remaining portion of sensing component 814 may be fully implanted inside wall 806 or partially implanted in wall 806 and having a portion 924 protruding into lumen 810 defined by wall 806.

Either one or both of inductance coil 920 and antenna 918 may also be incorporated in, or serve themselves as an anchor such as anchor portion 254 (FIGS. 8A, 8B and 8C), in which case the shape and dimensions of the anchor as well as the materials from which it is constructed may be dependent on the desired electronic characteristics of inductance coil 920 and/or antenna 918 and external unit 914.

Other means of remote powering of and/or communication with an implanted sensor such as sensing component 814 as known in the art may also be employed to communicate one or more hemodynamic parameters selected from a group of parameters including blood pressure, blood velocity, degree of blood oxygenation (i.e., blood oxygen saturation) and temperature within the lumen of the monitored target organ between sensing component 814 and external unit 914.

Sensing component 814 may be configured for continuous real-time measurements for prolonged periods, for example at least one week, optionally at least one month, optionally at least one year, optionally at least 5 years, optionally at least 10 years; while wirelessly transmitting measured and/or analyzed signals/data to a remote out-of-body receiver.

Referring now back to FIGS. 8A, 8B and 8C; optionally, additionally or alternatively, sensory implant 10 may include additional components such as a temperature sensing component 820, a gyroscope 822 and accelerometer 824 for orientation and movement measurements. Such measurements could be processed in order to mitigate unwanted artifacts in sensing component 814 readouts. Sensing component 814 may be encapsulated in a seamless, one-piece biocompatible sheathing which may act as a pressure transferring medium. The sheathing may be shaped to minimize turbulence in blood or other fluid flow within the lumen of the organ.

In an embodiment in accordance to the current system, device and method, Sensory implant 10 may include only passive components. In such a configuration no electrical circuitry is employed to digitize the acquired measurement values, which obviates the need for electrically driven components of sensory implant 10, namely rechargeable power storing component 812, signal processing component 816 and telemetry component 818. Sensing component 814 values may be read employing external antenna 918 by applying an excitation signal. Resonant frequency of sensory implant 10 passive elements, which may be dependent on its conductive properties, may be analyzed telemetrically using only external circuitry.

Sensory implant 10 may be operative to be arranged in a packed configuration that enables its accommodation inside the lumen, for example of the rigid elongated member 504 along its longitudinal axis X. In some embodiments, for example, sensory implant 10 may include an outer diameter of 2 mm or less, optionally 1 mm or less. In some embodiments, sensory implant may be 10 mm or less in length.

Reference is now made to FIGS. 10A and 10B which are a perspective and cross-section view simplified illustrations of an exemplary two-members type sensory implant 20, in accordance with exemplary embodiments of the present invention. Sensory implant 20 is shown implanted across an organ wall 1012, comprising a first member 1052 and a second member 1054 connected to each other by a resilient or semi-rigid retaining portion or wire 1058. First member 1052 may also include a sensing component such as pressure sensitive membrane 1018 and be positioned at least partially inside the organ lumen 1020. Second member 1054 including at least one of a telemetry component and inductive coil 1008 positioned at least partially outside the organ wall 1012. As shown in FIG. 10A, member 1054 may include a coil 1008 made of an electrically conductive material and be optionally electrically insulated. Coil 1008 may be coiled around a core 1010 and be employed as at least one of a telemetry antenna 918 (FIG. 9) and an inductive coil 920 (FIG. 9) for receiving inductive energy by remote power feeding to charge electrical rechargeable power storing component 812 (FIG. 8) as explained above.

FIG. 10B illustrates sensory implant 20 following implantation. First member 1052 is in lumen 1020 defined by wall 1012 of a body organ. Second member 1054 is outside wall 1012. Both members may be held in place by wire 1058, sandwiching and abutting wall 1012: first member 1052 against luminal surface 1014 and second member 1054 against external surface 1016 of wall 1012 thus serving as anchors anchoring device 250 in place. In this embodiment, first member 1052 may house sensing component 1014 and second member 1054 may include an induction coil 1008 for receiving inductive energy and telemetry component 818 (FIG. 8). Any one of portions 1052 and 1054 may include electrical rechargeable power storing component 812 (FIG. 8) and signal processing component 816 (FIG. 8).

Wire 1058 may be elongated having a tubular or any other appropriate geometric cross-section and be made of a rigid, semi-rigid or flexible material. Additionally and optionally, wire 1058 may be made of a resilient material having super elastic properties. The resilience along the longitudinal axis of retention portion 1058 may be limited by a selection of a suitable material to allow a predetermined limited level of axial movement of members 1052 and 1054 along the longitudinal axis of wire 1058 and preventing members 1052 and 1054 from becoming too distant from each other losing their function as anchors. The relative axial movement with respect to sensory implant 20 as derived from the on-going oscillatory movement of the organ (e.g., atrial wall) may allow periodic measurement of the contraction force of the atrium muscles and/or serve as generating/recharging mechanism in a power harvesting scheme.

Additionally and optionally, wire 1058 may include a mesh portion improving scar tissue growth into the mesh openings over time thus further fixing sensory implant 20 in place. As explained above, the physical characteristics of wire 1058 may be employed to harness and convert mechanical energy produced by pulsating (e.g., 0 on-going deformation and stretching-contracting behavior) muscular wall tissue in which a sensory implant, such as device 250 is implanted into electrical energy that may be stored, for example, in a capacitor.

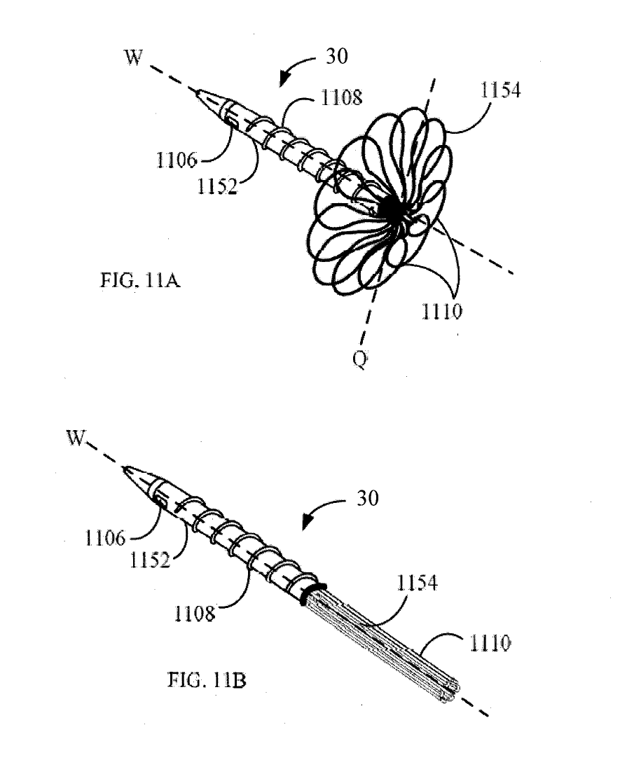

Reference is now made to FIGS. 11A and 11B, which are perspective view simplified illustrations of an exemplary flower-type sensory implant 30, in accordance with exemplary embodiments of the present invention. Sensory implant 30 includes an implanted or body portion 1152 and anchor portion 1154. Implanted portion 1152 may house one or more electrically driven components including an electrical rechargeable power storing component 812 (FIGS. 8A, 8B and 8C), a sensing component 814 and a signal processing component 816. Portion 1152 may also include a pressure sensitive member 1106, such as membrane 904 (FIG. 9) of sensing component 814. Portion 1152 may also include a spiral thread 1108 to provide greater fixation within the wall of the body organ to be monitored.

Anchor portion 1154 may be made of one or more resilient elements 1110 made of a material such as, for example a wire, textile or polymer mesh having super elastic properties to bring anchor portion 1154 from a temporary closed or packed loaded state shown in FIG. 11B to a relaxed open state shown in FIG. 11A by releasing stored potential energy. In some embodiments, anchor portion 1154 is made of a super-elastic alloy, for example a Ni—Ti alloy wire.

Alternatively and optionally, anchor portion 1154 may be made of a temperature sensitive material that changes its geometrical shape from that shown in FIG. 11B to that shown in FIG. 11A when exposed to a change in temperature. Once implanted, scar tissue may grow over time into gaps 1110 and/or partially or completely cover anchor 1154 further fixing sensory implant 30 in place.

Alternatively and optionally, anchor portion 1154 may be made of a mesh (not shown) expanded by employing an inflatable balloon on the implant delivery system as described above. Alternatively and optionally, anchor portion 1154 may be expanded when deployed by an umbrella-type expanding mechanism (not shown).

When in a closed or packed configuration (FIG. 11B), anchor portion 1154 may be capable of acquiring a spatial geometrical longitudinal shape along the longitudinal axis W of body portion 1152. Additionally, anchor portion 1154 may be capable of acquiring a diameter smaller than the diameter of enclosing lumen boundaries so that sensory implant 30 could fit inside and retain its ability to slidingly and axially move along lumen 206, making the length dimension of folded anchor portion 1154 longer than its diameter dimension.

In its deployed or expanded configuration (FIG. 11A), anchor portion 1154 may be capable of assuming a flower- or a canopy-like geometrical shape having a diameter, e.g., along axis Q, optionally at least 5 times greater than anchor portion 1154 diameter in its closed configuration (11B) as described above. Additionally, in the deployed or expanded configuration the axis (e.g., axis Q) of anchor portion 1154 diameter may be perpendicular or close to perpendicular to longitudinal axis W of sensory implant 30 body portion 1152. Alternatively and optionally, anchor portion 1154 may also serve as an antenna such as antenna 918 (FIG. 9) and/or an inductance coil such as inductance coil 920 (FIG. 9).

Alternatively and optionally, anchor portion 1154 may include any one of an inflatable chamber, a shape memory material (e.g., Ni—Ti based alloy), cage, mesh or spring type expandable mechanism selectively changeable by an operator from outside the body.

In some embodiments, additional optional elements such as ring-like elements, protrusions or indentations (not shown) may be provided and/or lateral or longitudinal geometrical cross-sections in various shapes may be applied to sensory implant 30 to enhance fixation and stabilization thereof in the wall.

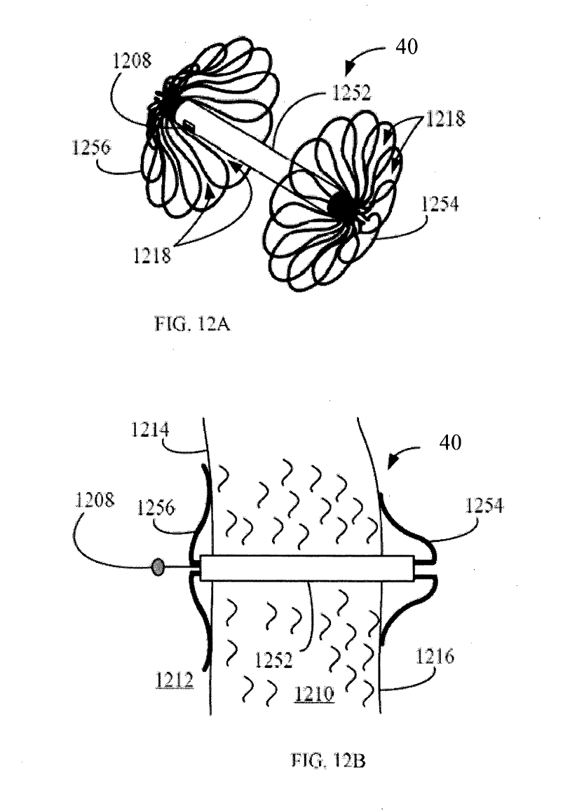

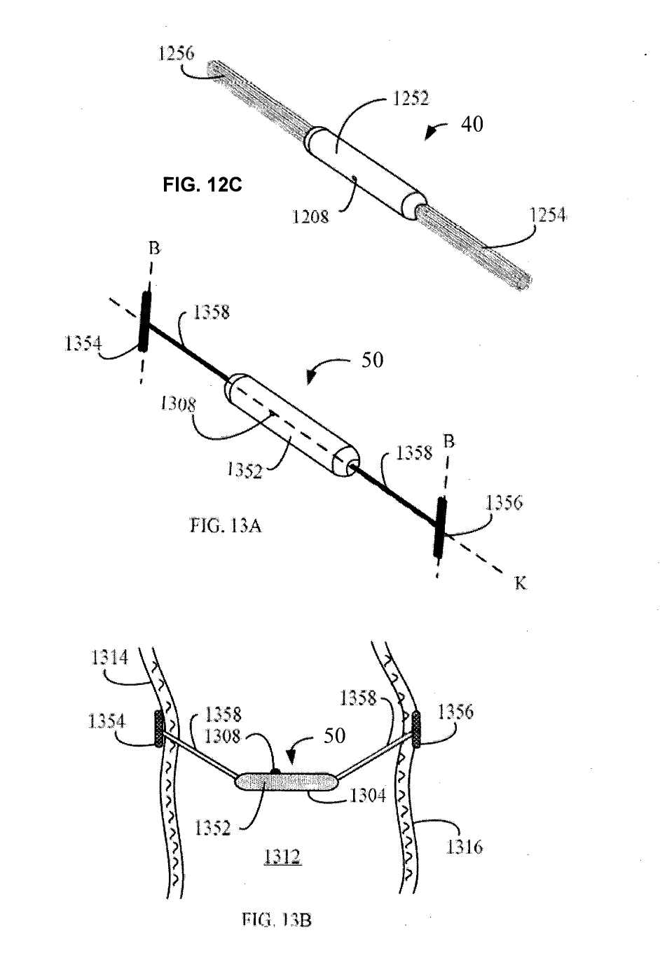

Reference is now made to FIGS. 12A, 12B and 12C which are perspective view and cross-section view simplified illustrations of an exemplary double-flower-type sensory implant 40, in accordance with exemplary embodiments of the present invention. As shown in FIG. 12A, sensory implant 40 includes an implanted or body portion 1252 and two or more anchor portions 1254 and 1256. Implanted or body portion 1252 may house or be one or more electrically driven components including an electrical rechargeable power storing component 812 (FIGS. 8A, 8B and 8C), a sensing component 814 and a signal processing component 816. Portion 1152 may also include a pressure sensitive member 1106, such as membrane 904 (FIG. 9) of sensing component 814. As illustrated in FIG. 12B, implanted portion 1252 may be implanted in tissue wall 1210, anchor portion 1256 may protrude into lumen 1212 defined by wall 1210 and anchor portion 1254 may protrude outside wall 1210. Both portions 1254 and 1256 may be held in place by implanted portion or body 1252, acting in a fashion similar to that of wire 1058 (FIGS. 10A and 10B), sandwiching and abutting wall 1210: anchor 1256 against luminal surface 1214 of wall 1210 and anchor 1256 against external surface 1216 of wall 1210.

Anchor portions 1254 and 1256 may be made of one or more resilient elements made of a material such as, for example a wire or mesh having super elastic properties to bring anchor portions 1254 and 1256 from a temporary closed loaded state shown in FIG. 12C to a relaxed open state shown in FIG. 12A as in a similar fashion described in FIGS. 11A and 11B. Alternatively and optionally, anchor portions 1254 and 1256 may be made of a temperature sensitive material that changes its geometrical shape from that shown in FIG. 12C to that shown in FIG. 12B when exposed to a change in temperature. The properties of anchor portions 1254 and 1256 may be the same as or similar to the properties of anchor portion 1154 described in FIGS. 11A and 11B.

Once implanted, scar tissue may grow over time into gaps 1218 and/or partially or completely cover anchors 1254 and 1256 further fixing sensory implant 40 in place. In such a case, pressure sensitive member 1208 may be covered with scar tissue interfering with its function. As shown in FIG. 12B, pressure sensitive member 1208 may be positioned in such a fashion so that to protrude at least 3 mm, optionally at least 5 mm, into lumen 1212, away from anchor portion 1256 and any scar tissue that may cover portion 1256 over time, keeping pressure sensitive member exposed to lumen 1212.

Reference is now made to FIGS. 13A and 13B which are perspective and cross-section view simplified illustrations of an exemplary hanging-type sensory implant 50, in accordance with exemplary embodiments of the present invention. In this embodiment, sensory implant 50 may be suspended in a lumen 108 of a body organ such as shown in FIG. 1C. Portion 1352 may house or be one or more electrically driven components including an electrical rechargeable power storing component 812 (FIGS. 8A, 8B and 8C), a sensing component 814 and a signal processing component 816. Portion 1352 may also include a pressure sensitive member 1308, such as membrane 904 (FIG. 9) of sensing component 814 (FIGS. 8A, 8B and 8C). Anyone of portions 1352, 1354 and 1356 may include or serve as an antenna such as antenna 918 (FIG. 9) and/or an inductance coil such as inductance coil 920 (FIG. 9).

Sensory implant 50 may be held suspended in lumen 1312 defined by walls 1314 and 1316, by two or more retention portions 1358 and two or more anchors 1354 and 1356, abutting respective walls 1314 and 1316.

Retention portions 1358 may be elongated having a tubular or any other appropriate geometric cross-section and be made of a rigid, semi-rigid or flexible material. Additionally and optionally, portion 1358 may be made of a resilient material having super elastic properties. The resilience along the longitudinal axis of retention portion 1358 may be limited by a selection of a suitable material to allow a predetermined limited level of freedom of axial movement of anchors 1354 and 1356 along the longitudinal axis of portions 1358 and preventing anchors 1354 and 1356 from becoming too distant from each other losing their function.

Sensory implant 50 as shown in FIG. 13A is in a deployed configuration in which the longitudinal axis B of one or more anchors 1354 and 1356 may be capable of assuming a position in 3D space perpendicular or close to perpendicular to longitudinal axis K of body 1352 and retention portions 1358. Anchors 1354 and 1356 may not necessarily parallel each other. In the packed configuration, anchors 1354 and 1356 may be capable of acquiring an orientation parallel to longitudinal axis X of needle 504 lumen 502 (FIG. 5A). Additionally and optionally, one or more anchors 1354/1356 may be made of a mesh allowing scar tissue growth into the mesh pores over time thus further fixing sensory implant 50 in place. Alternatively and optionally, one or more anchors 1354/1356 may be made of one or more resilient elements made of a material such as, for example a wire or mesh having super elastic properties to bring anchors 1354/1356 from a temporary closed loaded state to a permanent relaxed open state by releasing stored mechanical energy similar to anchors 1154 shown in FIGS. 11A and 11B.

In some embodiments, any of the anchors described in the present invention may be operative to allow relative axial movement of the sensory implant relative to the organ wall so that to prevent tearing of the organ wall issue resulting from shearing forces exerted by a axial movement of the pulsating wall tissue against a stationary implanted body. Allowing relative axial movement of the sensory implant also diminishes forces exerted by the organ wall tissue thus reducing fatigue and lengthening the sensory implant longevity. Additionally, anchors such as anchor 1154 (FIGS. 11A and 11B) may be designed and made from resilient or other materials having minimal fatigue properties so they do not lose resilience over time and injuring organ wall (mainly pulsating wall) tissue.

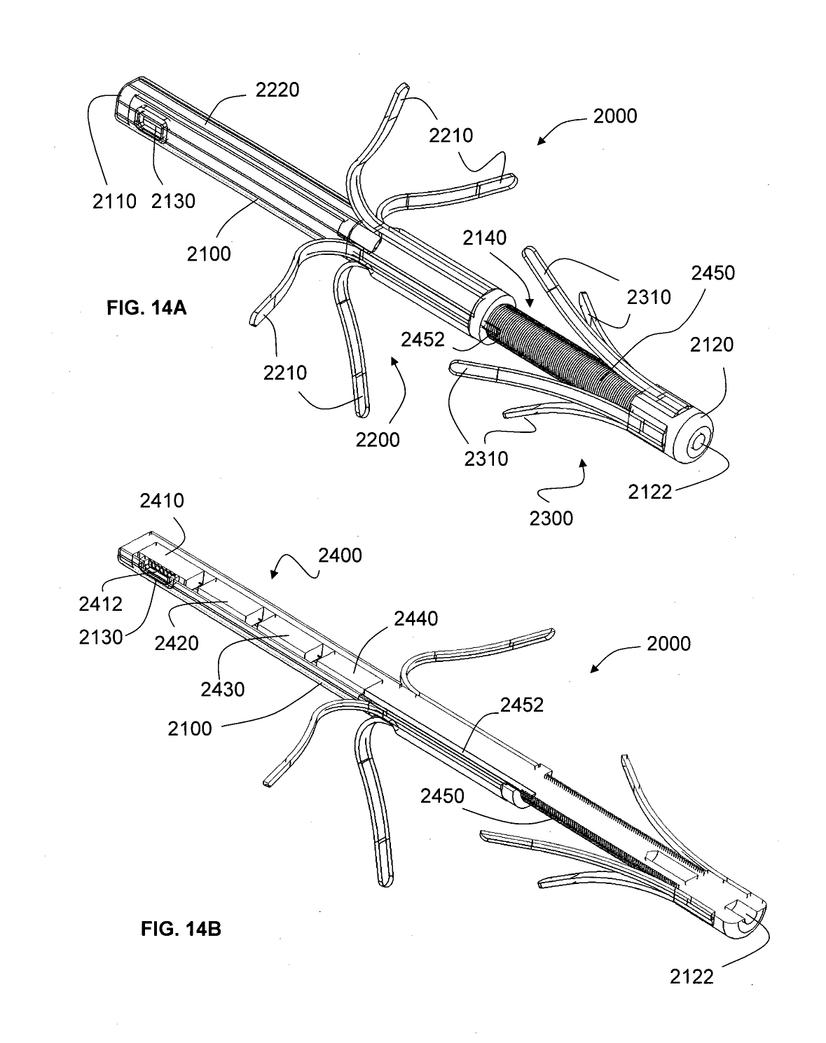

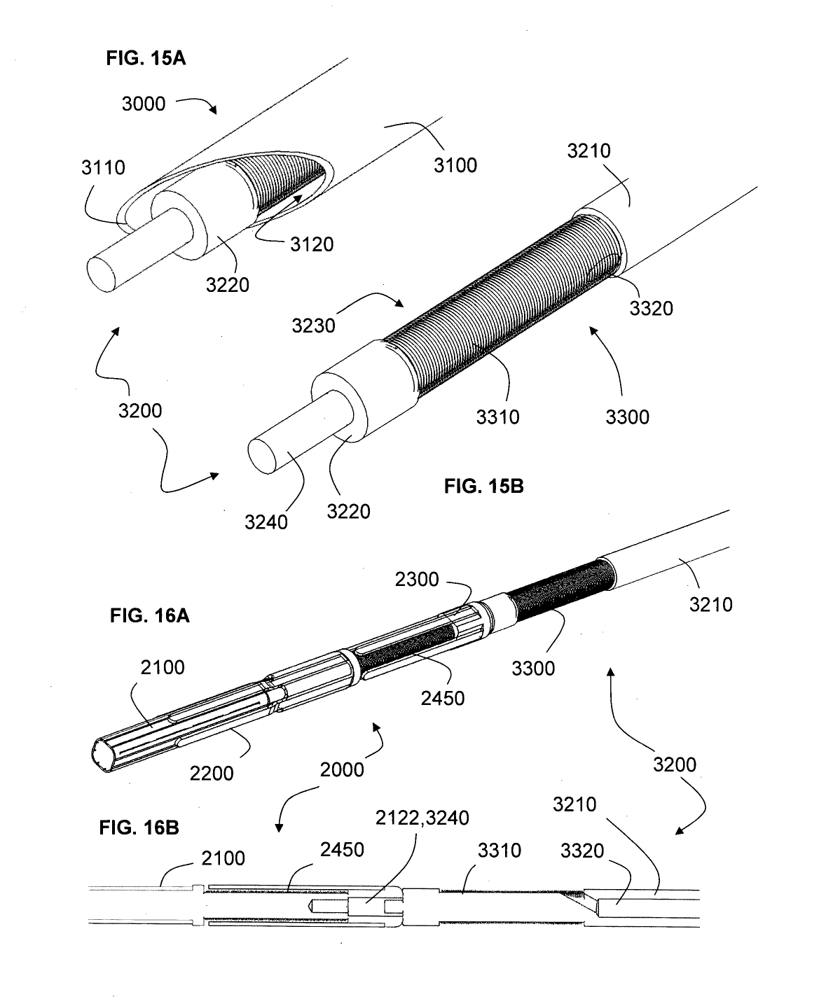

Reference is now made to FIGS. 14A and 14B which illustrate complete and transversally cut perspective views of an exemplary sensory implant 2000 (shown in a fully deployed formation) that is releasably connectable to an introducer 3300 (shown in FIGS. 15A and 15B hereinafter), in accordance with exemplary embodiments of the present invention. Sensory implant 2000 includes an elongated body or capsule 2100 extending between a distal end 2110 and a proximal end 2120. Provided (either coupled, welded, glued or otherwise fixated) along capsule 2100 length, are a distal expandable anchor 2200, positioned adjacent mid-length or closer to distal end 2110, and a proximal expandable anchor 2300, positioned adjacent proximal end 2120. Each of anchors 2200 and 2300 includes a plurality of retaining members (in this example, four retaining members each), 2210 and 2310, respectively, capable of elastically altering formation from a longitudinal form (in parallel to capsule 2100 axis) to an angled form, as shown in FIG. 14, thereby enlarging anchors transverse sizes. In case of implantation to a previously punctured or otherwise penetrated wall of an internal body organ (e.g., an atrial wall), anchors 2200 and 2300 are configured to expand to over the wall penetration diameter, thereby enabling sensory implant 2000 anchoring to the sandwiched wall. Depending on wall thickness and/or pulsatory motion, anchors 2200 and/or 2300 may correspondingly change in shape and/or expansion state while altering resistance forces applied to the wall. Distal retaining members 2210 fit in corresponding stripped recesses 2220 and comply to nest therein when at packed configuration; whereas proximal retaining members 2310 are forced into concentric recess 2140 at packed configuration. Concentric recess 2140 is shown also encircled with an inductor 2450 comprising a coiled conductive wire.

Capsule 2100 encapsulates juxtaposed electrical circuit 2400 in the following distal-to-proximal order:
1. A sensor 2410, which optionally includes a MEMS type pressure transducer comprising a membrane 2412 being provided in pressure communication with outer environment via opening 2130;
2. An optional capacitor 2420 (for example, in case that sensor 2410 is a capacitive MEMS transducer);
3. An optional added electrical component 2430, for example a telemetry unit, a motherboard, a memory, a battery, an amplifier, an antenna, a different sensor type, or other.
4. An application-specific integrated circuit (ASIC) 2440, adapted to convert the MEMS capacitance to a frequency-encoded signal, which is then wirelessly transmitted to a remote device located outside patient's body.

Electrical circuit 2400 is electrically connected to inductor 2450, optionally via a wire 2452, set to introduce induction to the circuit for powering thereof by a remote power source (not shown).

FIGS. 15A and 15B illustrate perspective views of an implant delivery device 3000 and introducer 3200 releasably connectable to sensory implant 2000, in accordance with exemplary embodiments of the present invention. Implant delivery device 3000, which may be similar or identical to any of the previously described delivery devices, shown partly in FIG. 15A, comprises a longitudinal body 3100 enclosing a lumen 3120 communicating with an open free end 3110. Introducer 3200, in part, is shown partly protruded through lumen 3120 in FIG. 15A and completely uncovered in FIG. 15B. Introducer 3200 includes an elongated body 3210 having a distal end 3220 and a concentric recess 3230 provided along a portion thereof adjacent distal end 3200. A male connector 3240 is concentrically and distally connected or engraved out of distal end 3220 and is adapted for releasably connecting with a mating female connector 2122 opening at proximal end 2120 of sensory implant 2000. Connector 3240 may include a threading or alternatively or additionally may include other fastening means, for example a peripherally expandable portion, a magnet, a snap lock or other. Concentric recess 3230 is provided encircled with a coiled conductive wire 3310 applicable for inductive coupling with inductor 2450 of sensory implant 2000, to thereby provide power to electrical circuit 2400 and/or any component thereof, at least during implant delivery and/or deployment. Alternative or additionally, induction unit 3000, comprising coiled conductive wire 3310, is used for inductively charging an optional battery in sensory implant 2000. Optionally, alternative or additionally, induction unit 3000 is or includes a transponder, a receiver and/or a transmitter for wirelessly communication signals with ASIC 2440. In some embodiments, coil 3310 is wired or otherwise directly electrically connectable to a proximal or a remote (out of patient's body) connector or power source, via a wire 3320.

FIGS. 16A and 16B illustrate perspective and cross-sectional views of sensory implant 2000 now shown interconnected with introducer 3200 in packed configuration, as if provided in conveying lumen 3120 boundaries in longitudinal body 3100, in accordance with exemplary embodiments of the present invention.

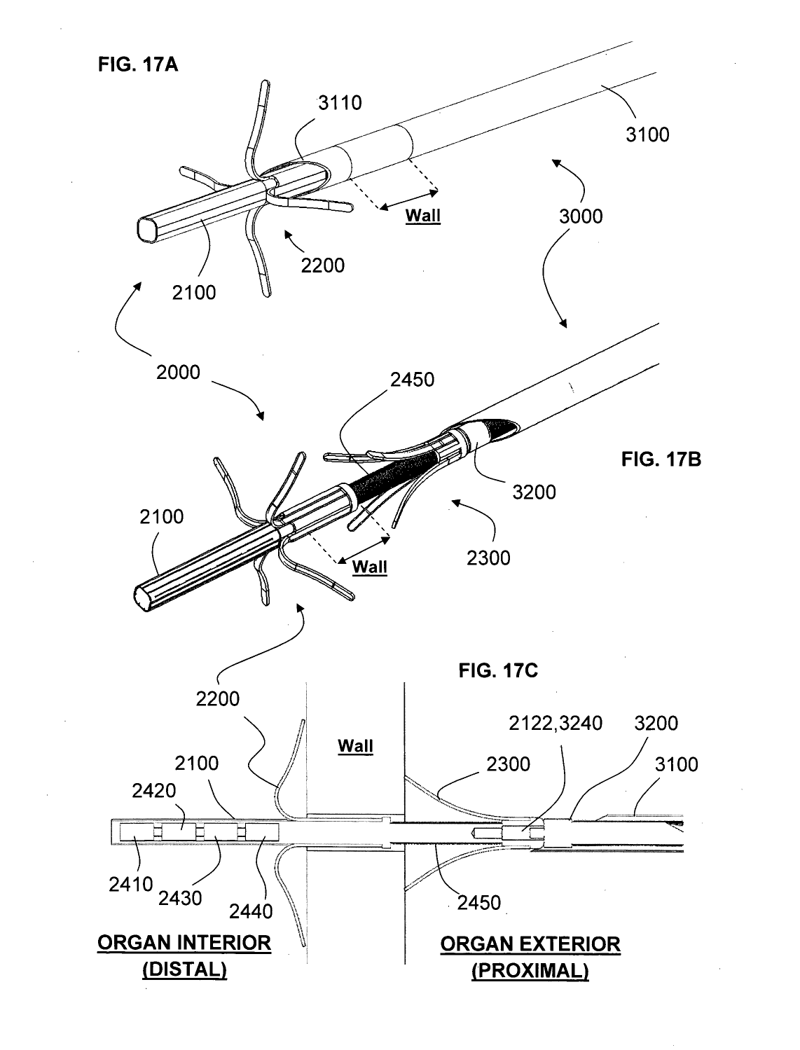

Reference is now made to FIGS. 17A, 17B and 17C which illustrate perspective and cross-sectional views of deployment stages of sensory implant 2000 in a wall of an internal body organ, in accordance with exemplary embodiments of the present invention. The organ wall is shown in cross section in FIG. 17C, whereas FIGS. 17A and 17B present virtual markings of a sandwiched portion thereof. In FIG. 17A, the system is shown after longitudinal body 3100 has penetrated through and over the wall and sensory implant 2000 been advanced forward (distally) using introducer 3200 to a partial protruded position whereby only distal anchor 2200 expands to a less/non-stressed expanded form whereas proximal anchor is maintained enclosed and confined to boundaries of lumen 3120. At this interim deploying phase the system operator may use some verification techniques and/or means to verify requested/correct positioning, alignment and/or protrusion depth in body organ interior. Optionally, alternatively or additionally, verification may include or followed with testing and/or calibrating sensory implant 2000 in view of data collected internally from the body organ.

In FIGS. 17B and 17C, a follow up step is shown, in which longitudinal body 3100 is retracted until proximal anchor 2300 fully emerges out and expands. This way, the wall is sandwiched between the retaining members of anchors 2200 and 2300. As shown, sensory implant 2000 is designed and sized such that sensor 2410 is substantially distant to internal surface of the wall at organ interior. In case that body organ tissue, emerging from the wall, shall react to the trauma (wall penetration) and/or foreign body presence (implanted sensory implant 2000) by covering it with tissue, the protrusion length of sensory implant 2000 in body organ interior may suffice to avoid covering sensor 2410.

At any deployment phase, at least until releasing sensory implant 2000, in case the operator wish to redeploy or totally discard sensory implant 2000, he may re-enclose it and re-collapse distal anchor 2200 and proximal anchor 2300 by pushing longitudinal body 3100 over it. As shown, both anchors 2200 and 2300 include retention members connected at proximal portions thereof and having free ends angled distally, thereby facilitating such complete recollapsing ability.

In some embodiments of the invention, a system is provided comprising sensory implant 2000 that is adapted to sense a change in at least one parameter associated with a condition and/or a performance of an internal body organ, optionally a cardiac left atrium. In some embodiments, the wall includes muscle tissue, optionally the wall is pulsatory. In some embodiments, sensory implant 2000 includes a sensing component, a signal processing component, and a telemetry component. In some embodiments, sensory implant 2000 includes a micro electro mechanical pressure transducer.

The system may further include an implant delivery device 3000 which includes longitudinal body 3100 enclosing a lumen 3120 communicating with an open free end 3110 thereof and adapted to provide a contained passage along a linear pierced path between an entry point and a wall target associated with the internal body organ. In some embodiments, the entry point is percutaneous or alternatively is adjacent an external surface of the wall (for example, during open surgeries). In some embodiments of the invention, the wall target is located on an external surface of the wall. Optionally and alternatively, the wall target is located in the wall. In some embodiments of the invention, the contained passage is of 2 mm or less, optionally 1.5 mm or less, optionally 1 mm or less in diameter, or higher or lower or intermediate.

Implant delivery device 3000 further includes introducer 3200 that is releasably connected to sensory implant 2000 and adapted to advance the sensory implant from an enclosed position in lumen 3120 to a protruded position distal to free end 3110. In some embodiments of the invention, introducer 3200 is coupled to sensory implant 2000 by a threaded mechanism. When connected to sensory implant 2000, introducer 3200 may allow electrical connectivity between sensory implant 2000 and a remote device provided outside the body (not shown), optionally including an electrical power source and/or a signal receiver. In some embodiments of the invention, introducer 3200 includes a distally positioned coiled member 3310 provided in a direct electrical contact with a proximally positioned electrically conductive member (not shown), the coiled member 3310 is adapted for inductive coupling with inductor 2450 provided with sensory implant 2000.

In some embodiments of the invention, implant delivery device 3000 is operative to penetrate the wall from its external surface and to release sensory implant 2000 in the wall.

In some embodiments of the invention, the linear pierced path is straight, optionally is at least 10 cm in length.

In some embodiments of the invention, sensory implant 2000 includes at least one retention member (2210 and/or 2310) capable of altering from a longitudinal form when confined to lumen 3120 boundaries, to an angled form when protruding out lumen 3120. In some embodiments, at least one of retention members 2210/2310 includes a resilient portion being at a higher stressed condition when retention member 2210/2310 is at the longitudinal form than when at the angled form. Optionally, the angled form is perpendicular to the longitudinal form. In some embodiments, anchor 2200 and/or anchor 2300 is configured to expand from a collapsed transverse size imposed by lumen 3120 boundaries to an expanded transverse size being 2 mm or more greater than a maximal diameter of free end 3110.

In some embodiments, inductor 2450 is expandable and configured to expand from a collapsed transverse size imposed by lumen 3120 boundaries to at least 5 mm in diameter when is unstressed. In some embodiments, an expandable inductor is further configured to perform as an anchoring device when expanding over the collapsed transverse size. Optionally, an expandable inductor includes an induction coil interlaced with an antenna coil.

In some embodiments of the invention, the system further includes a positioning verification analyzer communicative with the sensory implant prior to the releasing thereof and adapted to correlate between a signal transmitted by the sensory implant and a stored identifying data.

In some embodiments of the present invention, sensory implant 2000 is implantable in a muscular wall of a cardiac left atrium and is adapted to sense pressure changes developing inside the atrium. In some embodiments, sensory implant 2000 includes capsule 2100 being 1.5 mm or less in diameter, optionally 1 mm or less, optionally about 0.5 mm or less, or higher, or lower, or intermediate. In some embodiments, capsule 2100 contains a micro electro mechanical sensing component. In some embodiments, capsule 2100 is provided coupled with inductor 2450 and with an anchor (2200 and/or 2300) adapted to alter from a longitudinal form, equal to or less than the capsule diameter, to an angled form having a diameter greater than the lumen 3120 diameter. In some embodiments of the invention (not shown), a retaining member or an anchor includes and/or is interlaced with an inductor.

In an aspect of some embodiments the is provided a method which includes an introducing of implant delivery device 3000, carrying an implant, such as sensory implant 2000, along a selected linear pierced path and arriving at a wall target located on an external surface of a cardiovascular organ; and anchoring at least a portion of the implant in the cardiovascular organ wall.

In an aspect of some embodiments the is provided, in parallel, a method which includes an of

introducing implant delivery device 3000, carrying an implant, such as sensory implant 2000, along a selected linear pierced path and arriving at a wall target located on an external surface of an internal body organ; and anchoring at least a portion of the implant in the internal body organ wall. In some embodiments, the method further includes an implanting the implant in a site of implantation in the wall of the internal body organ; the site of implantation includes or is included in the wall target. In some embodiments, the method includes verifying implant location prior to the anchoring.

In some embodiments of the invention, the selected linear pierced path is a direct path that accommodates introduction and advancement of implant delivery device 3000 in a straight line. In some embodiments, delivery device 3000 passes at least once through lung tissue during its advancement along the linear pierced path. Optionally, the selected linear pierced path is selected prior to the introducing. Optionally, such a selecting is followed by a preliminary imagery scan analysis. In some embodiments, the method further includes selecting an entry point defining the linear pierced path in-between the organ target.

In some embodiments, the method includes a first step of selecting a wall target located on an external surface of an internal body organ, and then choosing a linear path between an entry point and the wall target. In some embodiments, the system or the implant delivery device, is used to pierce across tissues along the linear path. Optionally, sensory implant 2000 is advanced from an enclosed position in lumen 3120 to a protruded position distal to free end 3110. In some embodiments, the wall of the internal body organ is penetrated from the exterior surface. After penetration, following an optional verification phase, sensory implant 2000 is released in the wall.

It will be appreciated by those skilled in the art that the system, device and method described above may be used not just for the vascular system and may be applicable to other various organ types, body regions etc.

It will also be appreciated by persons skilled in the art that the present invention is not limited to what has been particularly shown and described hereinabove. Rather, the scope of the invention includes both combinations and sub-combinations of various features described hereinabove as well as modifications and variations thereof which would occur to a person skilled in the art upon reading the foregoing description and which are not in the prior art.

1. A system comprising:
(1) a sensory implant adapted to sense a change in at least one parameter associated with a condition and/or a performance of an internal body organ; and
(2) an implant delivery device, comprising:
(a) a longitudinal body enclosing a lumen communicating with an open free end thereof and adapted to provide a contained passage along a linear pierced path between an entry point and a wall target associated with the internal body organ; and
(b) an introducer releasably connected to the sensory implant and adapted to advance the sensory implant from an enclosed position in the lumen to a protruded position distal to the free end;
wherein the implant delivery device is operative to penetrate the wall from the exterior surface and to release the sensory implant in the wall.

2. A system according to claim 1, wherein the contained passage has a diameter of less than about 1.5 mm.

3. A system according to claim 1, wherein the introducer is coupled to the sensory implant by a threaded mechanism.

4. A system according to claim 1, wherein the introducer, when connected to the sensory implant, allows electrical connectivity between the sensory implant and a remote device provided outside the body; and
wherein the remote device includes an electrical power source and/or a signal receiver.

5. (canceled)

6. A system according to claim 4, wherein the introducer includes a distally positioned coiled member provided in a direct electrical contact with a proximally positioned electrically conductive member, the coiled member is adapted for inductive coupling with an implant conductor provided with the sensory implant.

7. A system according to claim 1, further comprising a positioning verification analyzer communicative with the sensory implant prior to the releasing thereof and adapted to correlate between a signal transmitted by the sensory implant and a stored identifying data.

8. A system according to claim 1, wherein the sensory implant includes a sensing component, a signal processing component, and a telemetry component.

9-28. (canceled)

29. A method comprising:
introducing an implant delivery device carrying an implant along a selected linear pierced path and arriving at a wall target located on an external surface of an internal body organ; and
anchoring at least a portion of the implant in the internal body organ wall.

30. The method of claim 29, further comprising: implanting the implant at a site of implantation in the wall of the internal body organ.

31. The method of claim 30, wherein the site of implantation includes the wall target.

32. The method of claim 29, wherein the implant is a sensory implant.

33. The method of claim 32, wherein the sensory implant includes a pressure sensor.

34. The method of claim 29, wherein the selected linear pierced path is a direct path accommodating introduction and advancement of the implant delivery device in a substantially straight line.

35. The method of claim 29, wherein the delivery device passes at least once through lung tissue during its advancement along the linear pierced path.

36. The method of claim 29, further comprising: verifying implant location prior to the anchoring.

37. The method of claim 29, wherein the selected linear pierced path is selected prior to the introducing.

38. The method of claim 37, wherein the selecting is followed by a preliminary imagery scan analysis.

39. The method of claim 29, further comprising: selecting an entry point defining the linear pierced path in-between the organ target.

40. The method of claim 29, wherein the implant delivery device comprises a longitudinal body enclosing a lumen communicating with an open free end thereof and adapted to provide a contained passage along the linear pierced path, and an introducer releasably connected to the sensory implant and adapted to advance the sensory implant in the lumen; and
wherein the method further comprises advancing the introducer from an enclosed position in the lumen to a protruded position distal to the free end.

41. A method comprising:
selecting a wall target located on an external surface of an internal body organ;
choosing a linear path between an entry point and the wall target;
using the system of claim 1 to pierce across tissues along the linear path;
penetrating the wall of the internal body organ from the external surface; and
releasing the sensory implant in the wall.

42. (canceled)
